(19) United States
(12) Patent Application Publication
(10) Pub. No.: US 2011/0318322 A1
(43) Pub. Date: Dec. 29, 2011

(54) Conjugates of a Lysosomal Enzyme Moiety and a Water Soluble Polymer

(75) Inventor: Mary J. Bossard, Madison, AL (US)

(73) Assignee: Nektar Therapeutics, San Francisco, CA (US)

(21) Appl. No.: 13/142,813

(22) PCT Filed: Jan. 12, 2010
(86) PCT No.: PCT/US2010/000078

§ 371 Date: Sep. 7, 2011

Related U.S. Application Data
(60) Provisional application No. 61/205,059, filed on Jan. 12, 2009.

(51) Int. Cl. A61K 47/48; A61P 3/00; C12N 11/08

(52) U.S. Cl. 424/94.3; 435/180

(57) ABSTRACT

Conjugates of a lysosomal enzyme moiety and one or more non-peptidic water soluble polymers are provided. Typically, the non-peptidic water soluble polymer is a poly(ethylene glycol) or a derivative thereof. Also provided, among other things, are compositions comprising such conjugates, methods of making the conjugates, and methods of administering the compositions to a patient, e.g., for treatment of a lysosomal storage disease.

### CROSS REFERENCE TO RELATED APPLICATION

This application claims the benefit of priority under 35 U.S.C. §119(e) to U.S. Provisional Patent Application Ser. No. 61/205,059, filed 12 Jan. 2009, the disclosure of which is incorporated herein by reference in its entirety.

### FIELD OF THE INVENTION

Among other things, the present disclosure relates generally to conjugates comprising a lysosomal enzyme moiety and a water soluble polymer. In addition, the disclosure relates to (among other things) compositions comprising such conjugates, methods of synthesizing and administering such conjugates, and methods for treating lysosomal storage disorders.

### BACKGROUND OF THE INVENTION

Lysosomal enzymes (acid hydrolases) are responsible for breaking down biologic macromolecules within the cell, more specifically, within an organelle within the cell called the lysosome. Such enzymes are found within the lysosome. More specifically, lysosomal enzymes degrade macromolecules and other materials that have been taken up by the cell during endocytosis by hydrolysis. The hydrolyzed products are then eliminated from the cell or reused. A deficiency of any one of these enzymes will usually lead to a “storage disease,” also referred to as a lysosomal storage disorder or LSD. The LSDs are a group of over 40 different disorders characterized by a lack of sufficient enzymatic activity to prevent the accumulation of specific macromolecules such as glycosphingolipids, mucopolysaccharides, or glycogen, in various tissues. Such storage diseases typically result in accumulation (“storage”) of substrates normally digested by a lysosomal protein within the cell, leading to enlargement of cells (ballooning), cellular disfunction, and eventually cell death. Lysosomal storage diseases are relatively rare, affecting one in every 100,000 to 200,000 infants. Each unique disorder is caused by a deficiency or dysfunction of a different enzyme. Signs of a lysosomal storage disease in infants or children may include growth failure, developmental regression, corneal or lens clouding, hepato- and/or splenomegaly, coarsening facial features and skeletal abnormalities.

Lysosomal enzymes include α-fucosidase, α-galactosidase, α-iduronidase, α-mannosidase, α-neuraminidase, β-galactoisidase, β-glucosidase, β-glucuronidase, β-mannosidase, hexosaminidase A, laronidase, galsulfase (Naglazyme), idursulfase (Elaprase), sphingomyelinase, galactocerebrosidase, arylsulfatase A, glucocerebrosidase, glycosaminoglycan cleaving enzymes, α-glucosidase, and lysosomal proteases, among others.

Lysosomal storage diseases associated with a lysosomal enzyme deficiency include fucosidosis (α-fucosidase), Fabry disease (α-galactosidase), Hurler syndrome (MPS I, α-iduronidase), α-mannosidosis (α-mannosidase), sialidosis (α-neuraminidase), GM1 gangliosidosis (β-galactoisidase), Gaucher disease (β-glucosidase/glucocerebrosidase), Sly syndrome (MPS VII, (β-glucuronidase), β-mannosidosis (β-mannosidase), GM2 gangliosidosis (Tay-Sachs disease, hexosaminidase A), mucospolysaccharidosis (MPS I, laronidase), mucopolysaccharidosis VI (galsulfase), mucopolysaccharidosis II (idursulfase), Niemann-Pick disease (sphingomyelinase), Globoid cell leukodystrophy (Krabbe disease, galactocerebrosidase), methachromatic leukodystrophy (arylsulfatase A), mucopolysaccharidoses (glycosaminoglycan cleaving enzymes), glycoproteinoses (glycoprotein cleaving enzymes), glycogenosis type II (Pompe disease, α-glucosidase), and neuronal ceroid lipofuscinoses (lysosomal proteases), where the enzyme in parenthesis following the LSD indicates the primary enzyme deficiency most typically associated with the disease.

Enzyme replacement therapy (“ERT”) can provide a therapeutic intervention for treating these disorders, although treatment is typically lifelong. Alglucerase (Ceredase) and Imiglucerase (Cerezyme) are approved for treatment of Gaucher disease. Laronidase (Aldurazyme) is approved for treatment of Hurler and Hurler-Scheie forms of MPS I. Agalsidase Beta (Fabrazyme) is approved for treatment of Fabry disease. Galsulfase (Naglazyme) is approved for treatment of MPS VI. Alglucosidase Alfa (Myozyme) is approved for treatment of infantile-onset Pompe disease. Idursulfase (Elaprase) is approved for treatment of Hunter syndrome (MPS II).

β-Glucocerebrosidase (β-D-glucosyl-N-acylsphingosine glucohydrolase) is a lysosomal glycoprotein (molecular weight of about 60,500 Daltons) that catalyzes the hydrolysis of glucocerebroside (a glycolipid) to glucose and ceramide. In healthy humans, sufficient quantities of this important enzyme are produced such that glucocerebroside does not accumulate in certain cells in the body.

In individuals suffering from Gaucher disease, however, the gene controlling production of β-glucocerebrosidase is mutated. As a result of the mutation, insufficient levels of β-glucocerebrosidase are produced, and/or the enzyme that is produced fails to function properly. In any event, glucocerebroside is not adequately hydrolyzed and consequently accumulates in tissue macrophages. These tissue macrophages (typically located in the liver, spleen, and bone marrow) become engorged with the glycolipid. Clinical signs of a significant deficiency of β-glucocerebrosidase activity—which results in the accumulation of engorged tissue macrophages—include one or more of the following: an enlarged spleen; an enlarged liver; and skeletal complications.

Currently, patients suffering from Gaucher disease can be treated with enzyme replacement therapy. With ERT, patients suffering from Gaucher disease are administered an enzyme that has β-glucocerebrosidase activity. Commercially available forms of enzymes having β-glucocerebrosidase activity useful for treating individuals suffering from Gaucher disease include alglucerase (marketed under the CEREDASE® brand) and imiglucerase marketed under the (CEREZYME® brand), both of which are available from Genzyme Corporation (Cambridge, Mass.). With respect to imiglucerase, this enzyme is administered by intravenous infusion over one to two hours, typically from three times a week to once every two weeks.

The currently approved forms of ERT for treating individuals suffering from Gaucher disease and other LSDs such as Fabry, MPS I, Hurler, Scheie, MPS II, Hunter, MPS VI, Maroteaux-Lamy, and Pompe disease typically require dosing by infusion over a period of hours, accompanied by the supervision of a health care professional. In addition, ERT doses are generally administered relatively frequently, making ERT less than desirable for its patients.

### SUMMARY OF THE INVENTION

Accordingly, in one or more embodiments of the invention, a conjugate is provided, the conjugate comprising a lysosomal enzyme moiety covalently attached, either directly or through a spacer moiety, to a non-peptidic water-soluble polymer. The conjugate is typically provided as part of a composition such as a pharmaceutical composition.

In one or more embodiments of the invention, a conjugate is provided, the conjugate comprising a residue of a lysosomal enzyme moiety covalently attached through a hydrolytically stable linkage to a water-soluble polymer.

In one or more embodiments of the invention, a conjugate is provided, the conjugate comprising a residue of a lysosomal enzyme moiety covalently attached through a cleavable linkage to a water-soluble polymer.

In one or more embodiments of the foregoing, a conjugate comprising a residue of a lysosomal enzyme moiety covalently attached through a cleavable linkage to a water-soluble polymer is capable of cleavage under lysosomal conditions.

In one or more embodiments of the invention, a conjugate is provided, the conjugate comprising a residue of a lysosomal enzyme moiety covalently attached to a linear water-soluble polymer.

In one or more embodiments of the invention, a conjugate is provided, the conjugate comprising a residue of a lysosomal enzyme moiety covalently attached to a branched water-soluble polymer.

In one or more embodiments of the invention, a conjugate is provided, the conjugate comprising a residue of a lysosomal enzyme moiety having a side chain comprising a cysteine residue, wherein the cysteine residue is attached, either directly or through a spacer moiety comprised of one or more atoms, to a water-soluble polymer.

In one or more embodiments of the invention, a conjugate is provided, the conjugate comprising a residue of a lysosomal enzyme moiety covalently attached, either directly or through a spacer moiety comprised of one or more atoms, to a water-soluble polymer, wherein the lysosomal enzyme moiety is attached to the water-soluble polymer or spacer moiety via an amide linkage, with the proviso that the amide linkage is not part of a carbamate linkage.

In one or more embodiments of the invention, a conjugate is provided, the conjugate comprising a residue of a lysosomal enzyme moiety covalently attached, either directly or through a spacer moiety comprised of one or more atoms, to a water-soluble polymer, wherein the lysosomal enzyme moiety is attached to the water-soluble polymer or spacer moiety via a secondary amine linkage, with the proviso that the secondary amine linkage is not part of a carbamate linkage. In a preferred embodiment, the secondary amine linkage results from reaction with a water-soluble polymer reagent having a reactive aldehyde group.

In one or more embodiments of the invention, a conjugate is provided, the conjugate comprising a residue of a lysosomal enzyme moiety covalently attached, either directly or through a spacer moiety comprised of one or more atoms, to a water-soluble polymer, wherein the lysosomal enzyme moiety is attached to the water-soluble polymer or spacer moiety via a linker other than a linker comprising a hydrazide or hydrazone linkage.

In one or more embodiments of the invention, a conjugate is provided, the conjugate comprising a residue of a lysosomal enzyme moiety covalently attached, either directly or through a spacer moiety comprised of one or more atoms, to a water-soluble polymer, wherein the lysosomal enzyme moiety is attached to the water-soluble polymer or spacer moiety via a thioether linkage.

In one or more embodiments of the invention, a conjugate is provided, the conjugate comprising a residue of a lysosomal enzyme moiety covalently attached, either directly or through a spacer moiety comprised of one or more atoms, to a water-soluble polymer, wherein the lysosomal enzyme moiety is attached to the water-soluble polymer or spacer moiety via a disulfide linkage. Preferably, the disulfide linkage is absent a hydrazone moiety, e.g., within the linkage.

In one or more embodiments of the invention, a composition is provided, the composition comprising a plurality of conjugates, each conjugate comprised of a residue of a lysosomal enzyme moiety attached, either directly or through a spacer moiety comprised of one or more atoms, to a water-soluble polymer, wherein at least 50% of all conjugates in the composition have the residue of the lysosomal enzyme moiety attached, either directly or through a spacer moiety comprised of one or more atoms, to the N-terminal of the lysosomal enzyme moiety.

In one or more embodiments of the invention, a composition is provided, the composition comprising a plurality of conjugates, each conjugate comprised of a residue of a lysosomal enzyme moiety attached, either directly or through a spacer moiety comprised of one or more atoms, to a PEG molecule, wherein at least 50% of all conjugates in the composition are N-terminally monoPEGylated.

In one or more embodiments of the invention, a composition is provided, the composition comprising a plurality of conjugates, each conjugate comprised of a residue of a lysosomal enzyme moiety attached, either directly or through a spacer moiety comprised of one or more atoms, to a water-soluble polymer having a weight average molecular weight between greater than 5,000 Daltons to about 80,000 Daltons.

In one or more embodiments of the invention, a composition is provided, the composition comprising a plurality of conjugates, each conjugate comprised of a residue of a lysosomal enzyme moiety attached, either directly or through a spacer moiety comprised of one or more atoms, to a water-soluble polymer, wherein at least 75% of all conjugates in the composition have a residue of a lysosomal enzyme moiety attached, either directly or through a spacer moiety comprised of one or more atoms, to five or fewer water-soluble polymers.

In one or more embodiments of the invention, a composition is provided, the composition comprising a plurality of conjugates, each conjugate comprised of a residue of a lysosomal enzyme moiety attached, either directly or through a spacer moiety comprised of one or more atoms, to a water-soluble polymer, wherein at least 75% of all conjugates in the composition have a residue of a lysosomal enzyme moiety attached, either directly or through a spacer moiety comprised of one or more atoms, to three or fewer water-soluble polymers.

In one or more embodiments of the invention, a conjugate is provided as set forth above, absent a targeting moiety.

In one or more embodiments of the invention, a conjugate is provided, wherein the conjugate exhibits lysosomal enzyme activity when evaluated in an in vivo or in vitro model.

In one or more embodiments of the invention, a conjugate is provided, the conjugate corresponding to the following structure:

POLY″-(X²)_{b}-POLY′-(X¹)ₐ-(LEM)

wherein:

POLY″ is a second water-soluble polymer (preferably branched or straight);

POLY′ is a first water-soluble polymer;

X¹, when present, is a first spacer moiety comprised of one or more atoms;

X², when present, is a second spacer moiety comprised of one or more atoms;

(b) is either zero or one;

(a) is either zero or one; and

LEM is a residue of a lysosomal enzyme moiety.

Also provided in one or more embodiments are methods for treating a LSD by subcutaneously administering a conjugate as provided herein.

In one or more embodiments, the lysosomal enzyme moiety is a glucocerebrosidase moiety.

Additional embodiments of the present conjugates, compositions, methods, and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

These and other objects and features of the invention will become more fully apparent when read in conjunction with the following detailed description.

### DETAILED DESCRIPTION OF THE INVENTION

Before describing one or more embodiments of the present invention in detail, it is to be understood that this disclosure is not limited to the particular polymers, synthetic techniques, lysosomal storage enzyme moieties, and the like, as such may vary.

It must be noted that, as used in this specification and the intended claims, the singular forms “a,” “an,” and “the” include plural referents unless the context clearly dictates otherwise. Thus, for example, reference to “a polymer” includes a single polymer as well as two or more of the same or different polymers, reference to “an optional excipient” refers to a single optional excipient as well as two or more of the same or different optional excipients, and the like.

### DEFINITIONS

In describing and claiming one or more embodiments of the present invention, the following terminology will be used in accordance with the definitions described below.

“PEG,” “polyethylene glycol” and “poly(ethylene glycol)” as used herein, are interchangeable and encompass any nonpeptidic water-soluble poly(ethylene oxide). Typically, PEGs for use in accordance with the invention comprise the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced, e.g., during a synthetic transformation. Throughout the specification and claims, it should be remembered that the term “PEG” includes structures having various terminal or “end capping” groups and so forth. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

The terms “end-capped” and “terminally capped” are interchangeably used herein to refer to a terminal or endpoint of a polymer having an end-capping moiety. Typically, although not necessarily, the end-capping moiety comprises a hydroxy or C₁₋₂₀ alkoxy group, more preferably a C₁₋₁₀ alkoxy group, and still more preferably a C₁₋₅ alkoxy group. Thus, examples of end-capping moieties include alkoxy (e.g., methoxy, ethoxy and benzyloxy), as well as aryl, heteroaryl, cyclo, heterocyclo, and the like. It must be remembered that the end-capping moiety may include one or more atoms of the terminal monomer in the polymer [e.g., the end-capping moiety “methoxy” in CH₃—O—(CH₂CH₂O)ₙ— and CH₃(OCH₂CH₂)ₙ—]. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) to which the polymer is coupled, can be determined by using a suitable detector. Such labels include, without limitation, fluorescers, chemiluminescers, moieties used in enzyme labeling, colorimetric (e.g., dyes), metal ions, radioactive moieties, and the like. Suitable detectors include photometers, films, spectrometers, and the like. The end-capping group can also advantageously comprise a phospholipid. When the polymer has an end-capping group comprising a phospholipid, unique properties are imparted to the polymer and the resulting conjugate. Exemplary phospholipids include, without limitation, those selected from the class of phospholipids called phosphatidylcholines. Specific phospholipids include, without limitation, those selected from the group consisting of dilauroylphosphatidylcholine, dioleylphosphatidylcholine, dipalmitoylphosphatidylcholine, disteroylphosphatidylcholine, behenoylphosphatidylcholine, arachidoylphosphatidylcholine, and lecithin.

“Non-naturally occurring” with respect to a polymer as described herein, means a polymer that in its entirety is not found in nature. A non-naturally occurring polymer may, however, contain one or more monomers or segments of monomers that are naturally occurring, so long as the overall polymer structure is not found in nature.

The term “water soluble” as in a “water-soluble polymer” polymer is any polymer that is soluble in water at room temperature. Typically, a water-soluble polymer will transmit at least about 75%, more preferably at least about 95%, of light transmitted by the same solution after filtering. On a weight basis, a water-soluble polymer will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer is about 95% (by weight) soluble in water or completely soluble in water.

“Molecular mass” in the context of a water-soluble polymer of the invention such as PEG, refers to the nominal average molecular mass of a polymer, typically determined by size exclusion chromatography, light scattering techniques, MALDI, or intrinsic viscosity determination in water or organic solvents. Molecular weight in the context of a water-soluble polymer, such as PEG, can be expressed as either a number-average molecular weight or a weight-average molecular weight. Unless otherwise indicated, all references to molecular weight herein refer to the weight-average molecular weight. Both molecular weight determinations, number-average and weight-average, can be measured using chromatographic techniques. Other methods for measuring molecular weight values can also be used, such as the use of end-group analysis or the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values preferably less than about 1.2, more preferably less than about 1.15, still more preferably less than about 1.10, yet still more preferably less than about 1.05, and most preferably less than about 1.03. As used herein, references will at times be made to a single water-soluble polymer having either a weight-average molecular weight or number-average molecular weight; such references will be understood to mean that the single-water soluble polymer was obtained from a composition of water-soluble polymers having the stated molecular weight.

The terms “active,” “reactive” or “activated” when used in conjunction with a particular functional group, refers to a reactive functional group that reacts readily with an electrophile or a nucleophile on another molecule. This is in contrast to those groups that require strong catalysts or highly impractical reaction conditions in order to react (i.e., a “non-reactive” or “inert” group).

As used herein, the term “functional group” or any synonym thereof is meant to encompass protected forms thereof as well as unprotected forms.

“Not readily reactive”, with reference to a functional group present on a molecule in a reaction mixture, indicates that the group remains largely intact under conditions effective to produce a desired reaction in the reaction mixture.

The terms “spacer moiety,” “linkage” and “linker” are used herein to refer to a bond or an atom or a collection of atoms optionally used to link interconnecting moieties such as a terminus of a polymer segment and a glucocerebrosidase moiety or an electrophile or nucleophile of a glucocerebrosidase moiety. The spacer moiety may be hydrolytically stable or may include a physiologically hydrolyzable or enzymatically degradable linkage. Unless the context clearly dictates otherwise, a spacer moiety optionally exists between any two elements of a compound (e.g., the provided conjugates comprising a residue of lysosomal enzyme moiety and water-soluble polymer can be attached directly or indirectly through a spacer moiety).

“Alkyl” refers to a hydrocarbon chain, typically ranging from about 1 to 15 atoms in length. Such hydrocarbon chains are preferably but not necessarily saturated and may be branched or straight chain, although typically straight chain is preferred. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl as well as cycloalkylene-containing alkyl.

“Lower alkyl” refers to an alkyl group containing from 1 to 6 carbon atoms, and may be straight chain or branched, as exemplified by methyl, ethyl, n-butyl, i-butyl, and t-butyl.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8 carbon atoms. “Cycloalkylene” refers to a cycloalkyl group that is inserted into an alkyl chain by bonding of the chain at any two carbons in the cyclic ring system.

“Alkoxy” refers to an —OR group, wherein R is alkyl or substituted alkyl, preferably C₁₋₆ alkyl (e.g., methoxy, ethoxy, propyloxy, and so forth).

The term “substituted” as in, for example, “substituted alkyl,” refers to a moiety (e.g., an alkyl group) substituted with one or more noninterfering substituents, such as, but not limited to: alkyl, C₃₋₈ cycloalkyl, e.g., cyclopropyl, cyclobutyl, and the like; halo, e.g., fluoro, chloro, bromo, and iodo; cyano; alkoxy, lower phenyl; substituted phenyl; and the like. “Substituted aryl” is aryl having one or more noninterfering groups as a substituent. For substitutions on a phenyl ring, the substituents may be in any orientation (i.e., ortho, meta, or para).

“Noninterfering substituents” are those groups that, when present in a molecule, are typically nonreactive with other functional groups contained within the molecule.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

“Heteroaryl” is an aryl group containing from one to four heteroatoms, preferably sulfur, oxygen, or nitrogen, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom that is not a carbon. Preferred heteroatoms include sulfur, oxygen, and nitrogen.

“Substituted heteroaryl” is heteroaryl having one or more noninterfering groups as substituents.

“Substituted heterocycle” is a heterocycle having one or more side chains formed from noninterfering substituents.

An “organic radical” as used herein shall include alkyl, substituted alkyl, aryl, substituted aryl,

“Electrophile” and “electrophilic group” refer to an ion or atom or collection of atoms, that may be ionic, having an electrophilic center, i.e., a center that is electron seeking, capable of reacting with a nucleophile.

“Nucleophile” and “nucleophilic group” refers to an ion or atom or collection of atoms that may be ionic having a nucleophilic center, i.e., a center that is seeking an electrophilic center or with an electrophile.

The terms “releasable,” “cleavable” and “degradable” are used interchangeably herein to refer to a linkage or bond (typically a linkage or bond between the residue of the lysosomal enzyme moiety and non-peptidic polymer in a conjugate) that cleaves. The term “hydrolyzable” represents a particular type of cleavable linkage or bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond, typically a covalent bond, that is substantially stable in water, that is to say, does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

“Pharmaceutically acceptable excipient or carrier” refers to an excipient that may optionally be included in the compositions of the invention and that causes no significant adverse toxicological effects to the patient. “Pharmacologically effective amount,” “physiologically effective amount,” and “therapeutically effective amount” are used interchangeably herein to mean the amount of a polymer-(glucocerebrosidase) moiety conjugate that is needed to provide a desired level of the conjugate (or corresponding unconjugated glucocerebrosidase moiety) in the bloodstream or in the target tissue. The precise amount will depend upon numerous factors, e.g., the particular glucocerebrosidase moiety, the components and physical characteristics of the therapeutic composition, intended patient population, individual patient considerations, and the like, and can readily be determined by one skilled in the art, based upon the information provided herein.

“Multi-functional” means a polymer having three or more functional groups contained therein, where the functional groups may be the same or different. Multi-functional polymeric reagents of the invention will typically contain from about 3-100 functional groups, or from 3-50 functional groups, or from 3-25 functional groups, or from 3-15 functional groups, or from 3 to 10 functional groups, or will contain 3, 4, 5, 6, 7, 8, 9 or 10 functional groups within the polymer backbone.

The term “glucocerebrosidase moiety,” as used herein, refers to a moiety having human β-glucocerebrosidase activity, and, unless the context clearly dictates otherwise, also refers to any β-glucocerebrosidase precursor moiety (such as provided in SEQ ID NOs: 5 and 6). The glucocerebrosidase moiety will also have at least one electrophilic group or nucleophilic group suitable for reaction with a polymeric reagent. In addition, the term “glucocerebrosidase moiety” encompasses both the glucocerebrosidase moiety prior to conjugation as well as the glucocerebrosidase moiety residue following conjugation. As will be explained in further detail below, one of ordinary skill in the art can determine whether any given moiety has glucocerebrosidase activity. Proteins comprising an amino acid sequence corresponding to any one of SEQ ID NOs: 1 through 4 is a glucocerebrosidase moiety, as well as any protein or polypeptide substantially identical thereto, that can act as a catalyst for the cleavage of glucocerebroside. As used herein, the term “glucocerebrosidase moiety” includes such proteins modified deliberately, as for example, by site directed mutagenesis or accidentally through mutations. These terms also include analogs having from 1 to 6 additional glycosylation sites, analogs having at least one additional amino acid at the carboxy terminal end of the protein wherein the additional amino acid(s) includes at least one glycosylation site, and analogs having an amino acid sequence which includes at least one glycosylation site. The term includes both natural and recombinantly produced moieties.

The term “substantially identical” means that a particular subject sequence, for example, a mutant sequence, varies from a reference sequence by one or more substitutions, deletions, or additions, the net effect of which does not result in an adverse functional dissimilarity between the reference and subject sequences. For purposes of the present invention, sequences having greater than 95 percent identity, equivalent biological properties, and equivalent expression characteristics are considered substantially homologous. For purposes of determining identity, truncation of the mature sequence should be disregarded. Sequences having lesser degrees of identity, comparable bioactivity, and equivalent expression characteristics are considered substantial equivalents. Exemplary glucocerebrosidase moieties for use herein include those sequences that are substantially identical to SEQ ID NO: 1.

The term “fragment”, for example of a lysosomal storage enzyme, means any polypeptide having the amino acid sequence corresponding to a portion of a particular lysosomal storage enzyme such as a glucocerebrosidase moiety, and which has the biological activity of the lysosomal storage enzyme. Fragments include polypeptides produced by proteolytic degradation of a lysosomal storage enzyme as well as polypeptides produced by chemical synthesis by methods routine in the art. Enzymatic activity is typically measured, e.g., by enzymatic or inhibitory activity using cultured cell lines or tissue culture based methods.

Lysosomal conditions refer to conditions found within the lysosome. In general, lysosomal conditions may be reproduced in vitro and include a pH of about 4.5-5.5 as well as a reducing environment.

The term “patient,” refers to a living organism suffering from or prone to a condition that can be prevented or treated by administration of an active agent (e.g., conjugate), and includes both humans and animals.

“Optional” or “optionally” means that the subsequently described circumstance may or may not occur, so that the description includes instances where the circumstance occurs and instances where it does not.

“Substantially” means nearly totally or completely, for instance, 95% or greater of some given quantity.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

To treat a condition such as a lysosomal storage disease means to ameliorate one or more symptoms associated with the disease, preventing or delaying onset of the disease and/or lessening the severity of frequency of symptoms associated with the disease.

Overview

Enzyme replacement therapy has been used successfully to treat certain LSDs such as type 1 Gaucher disease and Fabry disease, among others, and has been shown to lead to significant improvement of the clinical manifestations in patients suffering from these conditions. Enzyme replacement therapy involves regular (typically weekly) infusions of enzyme into the circulation of a patient deficient in the enzyme. While ERT can provide effective therapy for a number of lysosomal storage disease disorders, it is not without its significant drawbacks.

Most severe are LSD infusion-associated adverse reactions. Such infusion-related reactions include upper respiratory tract infection, rash, and injection site reaction. Common infusion-related hypersensitivity reactions include flushing, fever, headache, and rash. Moreover, immunogenicity accompanying ERT presents yet another significant concern. Additionally, high doses are typically required in ERT, and are accompanied by slow response and the inability to recover a majority of the infused enzyme in the target tissues. Such losses are attributed to occurring during transit of the enzyme en route to the lysosome.

Attempts to address (e.g., by modification with polymers such as PEG), one or more of the foregoing challenges have also been accompanied by related challenges and other problems, such as loss of bioactivity, lack of specificity of polymer attachment, production of complicated and inseparable conjugate compositions, irreproducibility of conjugate compositions, inability of the enzyme or enzyme conjugate to target the lysosome, and the like. A conjugate as provided herein is capable of overcoming at least one, and preferably several, of the foregoing drawbacks of existing enzyme replacement therapy methods.

The Lysosomal Enzyme Moiety

Lysosomal enzymes are acid hydrolases found in the lysosome, which function to breakdown complex biomolecules. Several lysosomal enzymes are glycoproteins that contain one or more O— and/or N-linked oligosaccharide side chains. As described in the background, a deficiency in a particular lysosomal enzyme or the activity of such enzyme leads to a lysosomal storage disorder or disease (LSD). Each unique disorder is caused by a deficiency or disfunction of a different enzyme. For ease of reference, the following Table 1 provides an overview of some of the more common LSDs and the corresponding deficient lysosomal enzyme.

### Overview of Some of the More Common LSDs

TABLE 1

| Lysosomal Enzyme | LSD |
|---|---|
| α-fucosidase | fucosidosis |
| α-galactosidase-A | Fabry disease |
| ceramide trihexosidase) | |
| α-iduronidase | Hurler syndrome (most severe) |
| | mucopolysaccharidosis type 1; MPS I |
| | gargoylism |
| | subtypes: |
| | MPS I S (Scheie syndrome) |
| | MPS I H-S (Hurler-Scheie syndrome) |
| α-mannosidase | α-mannosidosis |
| β-mannosidase | β-mannosidosis |
| α-neuraminidase | sialidosis |
| | mucolipidosis type I |
| β-galactoisidase | GM1 gangliosidosis |
| β-glucosidase/glucocerebrosidase | Gaucher disease |
| MPS VII, β-glucuronidase | Sly syndrome |
| hexosaminidase A | GM2 gangliosidosis |
| | Tay-Sachs disease |
| laronidase | mucospolysaccharidosis, MPS I |
| recombinant human | |
| a-L-iduronidase | |
| idursulfase | mucopolysaccharidosis II |
| N-acetylgalactosamine 4-sulfatase | mucopolysaccharidosis VI |
| galsulfase | Maroteaux-Lamy syndrome |
| sphingomyelinase | Niemann-Pick disease |
| galactocerebrosidase | Globoid cell leukodystrophy Krabbe |
| | disease |
| arylsulfatase A | methachromatic leukodystrophy |
| glycosaminoglycan cleaving | mucopolysaccharidoses |
| enzymes | |
| glycoprotein cleaving enzymes | glycoproteinoses |
| α-glucosidase | glycogenosis type II |
| | Pompe disease |
| lysosomal proteases | neuronal ceroid lipofuscinoses |

Preferred lysosomal enzyme moieties for use in the conjugates provided herein include but are not limited to glucocerebrosidase (e.g., Cerezyme and Ceredase), laronidase (Aldurazyme), α-galactosidase-A (e.g., agalsidase beta or Fabrazyme), N-aceytlgalactosamine 4-sulfatase (e.g., galsulfase or Naglazyme), alpha-glucosidase (e.g., alglucosidase alpha or Myozyme), and iduronate-2-sulfatase (e.g., idursulfase or Elaprase). The foregoing lysosomal enzyme moieties are meant to encompass truncated versions, hybrid variants, peptide mimetics, biologically active fragments, deletion variants, substitution variants or addition variants that maintain at least some degree of the subject lysosomal enzymatic activity.

The foregoing enzyme moieties may be isolated from human sources, animal sources, and plant sources. Alternatively, there may be obtained from either non-recombinant methods or from recombinant methods. In many cases, the lysosomal enzyme may be obtained from a commercial source.

The lysosomal enzyme moiety can be expressed in bacterial (e.g., E. coli), mammalian (e.g., Chinese hamster ovary cells), and yeast (e.g., Saccharomyces cerevisiae) expression systems. The expression can occur via exogenous expression (when the host cell naturally contains the desired genetic coding) or via endogenous expression.

Although recombinant-based methods for preparing proteins can differ, recombinant methods typically involve constructing the nucleic acid encoding the desired polypeptide or fragment, cloning the nucleic acid into an expression vector, transforming a host cell (e.g., plant, bacteria, yeast, transgenic animal cell, or mammalian cell such as Chinese hamster ovary cell or baby hamster kidney cell), and expressing the nucleic acid to produce the desired polypeptide or fragment. Methods for producing and expressing recombinant polypeptides in vitro and in prokaryotic and eukaryotic host cells are known to those of ordinary skill in the art. Exemplary constructs for expressing lysosomal polypeptides are provided in International PCT Publication No. WO 2004/064750.

To facilitate identification and purification of the recombinant polypeptide, nucleic acid sequences that encode for an epitope tag or other affinity binding sequence can be inserted or added in-frame with the coding sequence, thereby producing a fusion protein comprised of the desired polypeptide and a polypeptide suited for binding. Fusion proteins can be identified and purified by first running a mixture containing the fusion protein through an affinity column bearing binding moieties (e.g., antibodies) directed against the epitope tag or other binding sequence in the fusion proteins, thereby binding the fusion protein within the column. Thereafter, the fusion protein can be recovered by washing the column with the appropriate solution (e.g., acid) to release the bound fusion protein. The recombinant polypeptide can also be identified and purified by lysing the host cells, separating the polypeptide, e.g., by size exclusion chromatography, ion-exchange chromatography, and so forth, and collecting the polypeptide. These and other methods for identifying and purifying recombinant polypeptides are known to those of ordinary skill in the art. In one or more embodiments of the invention, however, it is preferred that the lysosomal enzyme moiety is not in the form of a fusion protein.

Depending on the system used to express the desired lysosomal enzyme as well as the particular lysosomal enzyme itself, the lysosomal enzyme moiety can be unglycosylated or glycosylated and either may be used. That is, the lysosomal enzyme moiety can be unglycosylated or the lysosomal enzyme moiety can be glycosylated.

The lysosomal enzyme moiety can advantageously be modified to include one or more amino acid residues such as, for example, lysine, cysteine and/or arginine, in order to provide facile attachment of the polymer to an atom within the side chain of the amino acid. In addition, the lysosomal enzyme moiety can be modified to include a non-naturally occurring amino acid residue. Techniques for adding amino acid residues and non-naturally occurring amino acid residues are well known to those of ordinary skill in the art using protein engineering methodologies.

Specifically, a molecule may be modified if necessary by deletion of an amino acid and/or incorporation of one or more non-natural amino acid residues into the molecule. For example, in certain cases, at least the N-terminal amino acid (typically a methionine) is replaced with a non-natural amino acid. Alternatively, a non-natural amino acid may be incorporated at the penultimate position, in addition to the N-terminal amino acid being replaced with a non-natural amino acid, and possibly other non-natural amino acid incorporations in the molecule. Auxotrophic host cells may be used for assistance in incorporating non-natural amino acids into the molecule. Additionally, mutant transcription or translation machinery for assistance in incorporating non-natural amino acids may be employed. Exemplary means of mutant transcription machinery include mutant tRNA and/or mutant amino-acyl tRNA synthetase(s). In some embodiments, a chemical moiety is attached to one or more of the non-natural amino acids of the modified molecule. Advantageously, a non-natural amino acid or a cysteine amino acid can be added or replaced at a location in molecule at an area relatively distant from areas of the molecule necessary for activity. In this way, the resulting conjugates are more likely to retain relatively higher levels upon conjugation as non-peptidic, water-soluble polymer attachment occurs at a location or location distant for activity.

Several detailed methods for altering molecules, including proteins, are set forth in U.S. patent application Ser. Nos. 09/620,691, now abandoned; 10/851,564, pending as U.S. Publication No. 20040219488; Ser. No. 10/612,713, pending as U.S. Publication No. 20040058415; Ser. No. 11/094,625, pending as U.S. Publication No. 20050260711; Ser. No. 11/130,583, pending as U.S. Publication No. 20050287639; U.S. Pat. No. 7,139,665; and U.S. Pat. No. 6,586,207; all of which are hereby incorporated by reference in their entireties. Additionally, several issued U.S. patents discuss methods for calculating energy analysis for point mutations in molecules, including proteins, such as U.S. Pat. Nos. 6,188,965; 6,269;312; 6,708,120; 6,792,356; 6,801,861 and 6,804,611, all of which are hereby incorporated by reference in their entireties. Any of these referenced, or any other methods of altering, modifying or identifying molecules may be used.

In particular, the lysosomal enzyme moiety can advantageously be modified to include attachment of a functional group (other than through addition of a functional group-containing amino acid residue). For example, the lysosomal enzyme moiety can be modified to include a thiol group (e.g., via the addition of a cysteine residue into the lysosomal enzyme moiety and/or via replacement in the lysosomal enzyme moiety of a non-cysteine amino acid residue with a cysteine residue). In addition, the lysosomal enzyme moiety can be modified to include an N-terminal alpha carbon. In addition, the lysosomal enzyme moiety can be modified to include one or more carbohydrate moieties.

Certain exemplary lysosomal enzyme moieties are described in greater detail below.

Glucocerebrosidase Moiety

As previously stated, in one of the many embodiments provided herein, the conjugate generically comprises a glucocerebrosidase moiety covalently attached, either directly or through a spacer moiety, to a non-peptidic water-soluble polymer. As used herein, the term “glucocerebrosidase moiety” refers to the glucocerebrosidase moiety prior to conjugation as well as to the glucocerebrosidase moiety following attachment to a non-peptidic water-soluble polymer. It will be understood, however, that when the original glucocerebrosidase moiety is attached to a non-peptidic water-soluble polymer, the glucocerebrosidase moiety is slightly altered due to the presence of one or more covalent bonds associated with the linkage to the polymer. Often, this slightly altered form of the glucocerebrosidase moiety attached to another molecule is referred to a “residue” of the glucocerebrosidase moiety. The glucocerebrosidase moiety in the conjugate is any peptide that provides β-glucocerebrosidase activity. The foregoing similarly applies to all other lysosomal enzyme moieties described herein.

The glucocerebrosidase moiety can be derived non-recombinantly. For example, as described in U.S. Pat. No. 3,910,822, it is possible to isolate glucocerebrosidase from human placental tissue. As provided therein, the process requires the steps of suspending the human placental tissue in a solvent, centrifuging the suspension, resuspending the centrifuged product, homogenizing the resuspended product and then purifying. The process results in a relatively pure glucocerebrosidase composition.

The glucocerebrosidase moiety can also be derived from recombinant methods. For example, International Patent Publication WO 92/13067 and U.S. Pat. No. 5,236,838 each describe recombinant-based methods for producing enzymatically active human glucocerebrosidase.

In one or more embodiments of the invention, it is preferred that the glucocerebrosidase moiety is glycosylated, preferably at four glycosylation sites. For example, it is also preferred to have the oligosaccharide chain at each glycosylation site terminate in a mannose sugar.

In some embodiments of the invention, it is preferred that the glucocerebrosidase moiety is not modified to include a thiol group and/or an N-terminal alpha carbon.

Preferred glucocerebrosidase moieties include those having an amino acid sequence comprising sequences selected from the group consisting of SEQ ID NOs: 1 through 4, and sequences substantially identical thereto. A preferred glucocerebrosidase moiety has the amino acid sequence corresponding to imiglucerase (Cerezyme™). Another preferred glucocerebrosidase has the amino acid sequence corresponding to alglucerase (Ceredase™). Another preferred glucocerebrosidase has the amino acid sequence corresponding to human placental glucocerebrosidase.

In addition, precursor forms of a protein that has β-glucocerebrosidase activity can be used. For example, a sequence corresponding to a “long isoform” is provided as SEQ ID NO: 5 and a sequence corresponding to a “short isoform” is provided as SEQ ID NO: 6, each of which can be used a glucocerebrosidase moiety herein.

A glucocerebrosidase moiety is meant to encompass truncated versions, hybrid variants, and peptide mimetics of any of the foregoing the sequence. Biologically active fragments, deletion variants, substitution variants or addition variants of any of the foregoing that maintain at least some degree of glucocerebrosidase activity can also serve as a glucocerebrosidase moiety.

For any given peptide or protein moiety, it is possible to determine whether that moiety has glucocerebrosidase activity. For example, as described in U.S. Pat. No. 5,236,838 (which references Methods of Enzymology, Vol. L, pp. 478-79, 1978), β-glucocerebrosidase activity can be determined using 4-methyl-umbelliferyl-B-D glucoside as a substrate. A moiety of interest can serve as a glucocerebrosidase moiety in accordance herein if a spectrofluorometer detects a fluorescent product resulting from enzymatic hydrolysis of 4-methyl-umbelliferyl-B-D glucoside. Another suitable assay for detecting lysosomal glucocerebrosidase activity is described in Chan et al. (2004) Analytical Biochemistry 334(2):227-233. Such assay measures β-glucocerebrosidase activity in equivalent soluble fluorophore units within Kupffer cell populations as defined by phenotype-specific monoclonal antibodies.

Laronidase Moiety

Yet another lysosomal enzyme moiety for use in the conjugates provided herein is a laronidase moiety. As with all of the lysosomal enzyme moieties described herein, such enzymes may be isolated from naturally occurring sources, may be synthesized either recombinantly or non-recombinantly by methods well known by those skilled in the art, or may be obtained from a commercial source. The term, “laronidase” or “laronidase moiety” is used herein to encompass any glycoprotein having α-L-iduronidase activity, regardless of its method of manufacture or slight differences in protein structure, as is the case for all other lysosomal enzyme moieties provided herein. Exemplary laronidase moiety sequences are described below.

Laronidase is a glycoprotein with a molecular weight of approximately 83 kilodaltons (Genbank Accession Number NP_{—}000194; also see Entrez GeneID No. 3425). The naturally occurring protein suitable for use as a laronidase moiety, α-L-iduronidase, is one of a series of ten lysosomal enzymes involved in the sequential degradation of glycosaminoglycans. Specifically, α-L-iduronidase catalyzes the hydrolysis of terminal α-L-iduronic acid residues of dermatan sulfate and heparin sulfate. Endogenous human α-L-iduronidase is synthesized in the endoplasmic reticulum as a 653 amino acid polypeptide and is glycosylated with six N-linked oligosaccharides to produce a 74 kilodalton precursor molecule. See Brooks et al. (2001) Glycobiology 11(9):741-750; Scott et al. (1992) Genomics 13(4):1311-1313, for the structure and sequence of the human alpha-L-iduronidase gene. The naturally occurring protein is one example of a laronidase moiety for use in the subject conjugates.

Alternatively, a recombinant laronidase moiety may be employed. The predicted amino acid sequence of the recombinant form (Aldurazyme®, laronidase-rch), as well as the nucleotide sequence that encodes it, are identical to human α-L-iduronidase. The recombinant protein contains 628 amino acids after cleavage of the N terminus and contains 6 N-linked oligosaccharide modification sites. The full length, glycosylated laronidase protein is produced by a genetically engineered Chinese hamster ovary cell line that has been transfected with the alpha-L-iduronidase cDNA coding region. See, e.g., Kakkis et al. (1994) Protein Expr Purif 5:225-232, as well as Drug Bank ID No. DB00090 for the protein sequence of human recombinant alpha-L-iduronidase, or laronidase. Recombinantly prepared laronidase is also suitable for use as a laronidase moiety in the conjugates provided herein.

One preferred laronidase moiety has an amino acid sequence corresponding to that of Aldurazyme®, human recombinant alpha-L-iduronidase, available from Biomarin Pharmaceuticals (Novato, Calif.). Aldurazyme® is marketed for the treatment of MPS I (Hurlers syndrome).

A laronidase moiety is meant to encompass truncated versions, hybrid variants, and peptide mimetics of any of the foregoing the sequences. Biologically active fragments, deletion variants, substitution variants or addition variants of any of the foregoing that maintain at least some degree of laronidase activity can also serve as a laronidase moiety.

The position of covalent attachment of a non-peptidic water soluble polymer to a laronidase moiety is preferably such that the enzymatic activity associated with the E182 and E299A residues (Brooks, ibid) is not adversely impacted. Laronidase activity may be determined, e.g., using 4-methylumbelliferyl iduronic acid as substrate (Hopwood et al. (1982) Clin Sci (Lond). 62:193-201.

α-Galactosidase-A Moiety (gla)

Yet another exemplary and preferred lysosomal enzyme moiety is alpha-galactosidase.

Alpha-galactosidase (an alpha-galactosidase moiety) (GenBank Acc. No. Genbank Accession Number NP_{—}000160; also see Entrez GeneID No. 2717) hydrolyzes the terminal alpha-galactosyl moieties from glycolipids and glycoproteins. See Bishop et al. (1986) PNAS 83(13):4859-4863 for the endogenous human sequence, suitable for use as an alpha-galactosidase moiety. As stated previously, an alpha-galactosidase moiety may be isolated from natural sources. The alpha-galactosidase A gene (GALA) has been shown to contain a number of polymorphisms in the first exon (Davies et al. (1993) J. Med. Genet. 30(8): 658-663). In terms of its function, alpha-galactosidase predominantly hydrolyses ceramide trihexoside, and can also catalyze the hydrolysis of melibiose into galactose and glucose. An alpha-galactosidase moiety will possess a degree of alpha-galactosidase activity prior to conjugation to a water soluble polymer.

Alternatively, an alpha-galactosidase moiety may be prepared using recombinant techniques. A particularly preferred alpha-galactosidase moiety corresponds to the recombinant human alpha-galactosidase sold under the tradename, Fabrazyme™ (agalsidase beta, Genzyme, Framingham, Mass.). Fabrazyme™ is recombinant human alpha-galactosidase A enzyme and possesses the same amino acid sequence as the native enzyme. Fabrazyme is a homodimeric glycoprotein, and is produced by recombinant DNA technology in a Chinese hamster ovary mammalian cell expression system. Fabrazyme™ is approved for treatment of Fabry disease (also referred to as Anderson-Fabry disease). Yet another preferred source of alpha-galactosidase is Replagal™ (agalsidase alfa, Shrire PLC). Both marketed enzyme therapeutics comprise alpha-galactosidase, but produced using different expression systems. Replagal™ is produced using a human cell line. Both forms are suitable for conjugation to a water soluble polymer as described herein. Both forms of an alpha-galactosidase moiety have similar glycosylation, both in the type and location of their oligosaccharides structures (Lee, K., et al., Glycobiology, 2003, 13 (4), 305-313). The enzymes differ in the ratio of oligomannose to complex oligosaccharides at two of the three N-linked glycosylation sites and also in the levels of terminal sugar residues, with Fabrazyme™ having a higher percentage of phsophorylated oligomannose chains and a higher percentage of fully sialylated complex oligosaccharides. (Lee, K., 2003, ibid). Polymer conjugates of alpha-galactosidase will preferably possess exposed mannose-6-phosphate residues to facilitate uptake by the mannose-6-phosphate receptor.

Subjects with Fabry disease possess a defect in the gene for alpha-galactosidase which results in an inability or diminished ability to catabolize lipids having terminal α-galatosyl residues. Such lipids, and in particular, globotriaosylceramide (GL-3) accumulate progressively in the lysosomes. Progressive pathologic changes in the kidney associated with Fabry disease typically result in renal failure by midlife in most classical cases of the disease.

Data suggest that the C-terminal region of the enzyme plays an important role in regulation of enzyme activity (Miyamura et al. (1996) J. Clin. Invest. 98(8):1809-1817); modifications and/or covalent attachment of a water soluble polymer to an alpha-galactosidase moiety will preferably take place at a site or in such a way as to maintain or enhance activity of the alpha-galactosidase A moiety by allowing access to its C-terminal.

Enzymatic activity of an α-galactosidase-A moiety or its corresponding polymer conjugate may be determined, e.g., using a spectrophotometric stop rate determination (Borooh et al. (1961) Biochemical Journal 78:106-110), or other suitable in vitro or in vivo assay. Receptor binding can be evaluated, e.g., using surface plasmon resonance to measure the interaction of alpha-galactosidase A with immobilized bovine soluble cation independent mannose-6-phosphate receptor (sCIMPR)

N-aceytlgalactosamine 4-sulfatase (e.g., Arylsulfatase B, Galsulfase or Naglazyme™)

Yet another exemplary and preferred lysosomal enzyme moiety is an N-aceytlgalactosamine 4-sulfatase moiety.

An N-aceytlgalactosamine 4-sulfatase (e.g., arylsulfatase B) moiety is a lysosomal enzyme moiety capable of catalyzing the cleavage of the sulfate ester from terminal N-acetylgalactosamine-4-sulfatase residues of glycosaminoglycans chondroitin 4-sulfate and dermatan sulfate. Specifically, an N-aceytlgalactosamine 4-sulfatase moiety is a lysosomal hydrolase capable of catalyzing the cleavage of the sulfate ester from terminal N-acetylgalactosamine 4-sulfate residues of glycosaminoglycans (GAG), chondroitin 4-sulfate and dermatan sulfate. A N-aceytlgalactosamine 4-sulfatase (e.g., arylsulfatase B) moiety may possess, e.g., an amino acid sequence corresponding to the native human sequence of N-aceytlgalactosamine 4-sulfatase (GenBank Accession No. NP_{—}000037; also see Entrez GeneID No. 411).

In one embodiment, an N-aceytlgalactosamine 4-sulfatase moiety is a recombinant N-aceytlgalactosamine 4-sulfatase moiety. In a preferred embodiment, an N-aceytlgalactosamine 4-sulfatase moiety will have a structure corresponding to that of Nalglazyme™. Nalglazyme™ is a recombinant version of N-aceytlgalactosamine 4-sulfatase marketed by BioMarin Pharmaceuticals (Novato, Calif.) for treatment of MPS VI.

Naglazyme™ is a normal variant of N-aceytlgalactosamine 4-sulfatase, produced by recombinant DNA technology in a Chinese hamster ovary cell line. See DrugBank ID No. 01279. Naglazyme™ is a single chain glycoprotein having a molecular weight of approximately 56 kD after cleavage of the signal peptide. The recombinant protein contains 495 amino acids and six asparagine-linked glycosylation sites. Four of the glycosylation sites carry a bis mannose 6-phosphate mannose oligosaccharide for specific cellular recognition. Naglazyme™ has eight cysteine residues, all of which are linked by intermolecular disulfide bridging. Post-translational modification of Cys53 produces the catalytic amino acid residue, Cα-formylglycine, which is required for enzyme activity and is conserved in al members of the sulfatase enzyme family. Methods for preparing and purifying a N-aceytlgalactosamine 4-sulfatase moiety are described in U.S. Pat. No. 6,972,124.

The enzymatic activity of an N-aceytlgalactosamine 4-sulfatase moiety, either prior to or after covalent attachment to a water soluble polymer to form a conjugate, can be assessed using, e.g., a specific and highly sensitive 4-sulfated trisaccharide-based assay of enzyme activity in fibroblasts (Brooks et al. (1991) Am J Hum. Genet. 48(4):710-719). Enzyme activity and lysosomal targeting receptor binding, e.g., of either a N-aceytlgalactosamine 4-sulfatase moiety or its corresponding conjugate, may also be assessed using a mannose-6-phosphate receptor-based in vitro assay as described in Kleinig et al. (1998) Analytical Biochemistry 260(2):128-134.

As described above, Naglazyme™ is used for treating MPS VI. Subjects suffering from MPS VI (Maroteaux-Lamy syndrome) are unable to produce or produce reduced amounts of N-aceytlgalactosamine 4-sulfatase. Patients suffering from MPS VI exhibit accumulation of dermatan sulfate throughout the body, leading to widespread and progressive cellular, tissue, and organ dysfunction. Clinical manifestations include short stature, kyphosis, coarse facial features, dysostosis multiplex, joint stiffness, heart valve thickening, upper airway obstruction, hepatosplenomegaly, and corneal clouding. The lifespan of most patients is reduced to between childhood and early adulthood.

Alpha-glucosidase (e.g., Acid Maltase, Alglucosidase Alpha or Myozyme™)

Yet another exemplary and preferred lysosomal enzyme moiety is an acid alpha-glucosidase moiety.

An acid alpha-glucosidase moiety is a lysosomal enzyme moiety that functions to degrade glycogen to glucose in lysosomes. Specifically, an acid alpha glucosidase moiety hydrolyzes both alpha-1,4- and alpha-1-6-glucosidic linkages and is essential for normal muscle development. Deficiency of the naturally occurring enzyme leads to accumulation of glycogen in lysosomes and cytoplasm, resulting in tissue destruction. Additionally, different forms of acid alpha-glucosidase exist due to proteolytic processing that occurs in the body.

An exemplary acid alpha-glucosidase moiety will have the amino acid sequence corresponding to NCBI GenBank Accession No. NP_{—}001073272. (Also see Entrez GeneID No. 2548 (human, endogenous protein).) Endogenous lysosomal alpha-glucosidase possesses seven glycosylation sites (Hermans et al. (1993) Biochem J. 289(Pt. 3):681-686). The sites at Asn-882 and Asn-925 are located in a C-terminal propeptide which is cleaved off during maturation. At least two of the oligosaccharide side chains of human lysosomal alpha-glucosidase are phosphorylated. Removal of the second glycosylation site at Asn-233 was found to interfere dramatically with the formation of mature enzyme.

An exemplary acid alpha-glucosidase moiety can be prepared recombinantly. In a preferred embodiment, an acid alpha-glucosidase moiety corresponds to recombinant acid alpha-glucosidase sold under the tradename, Myozyme™ (Genzyme, Framingham, Mass.). Myozyme™ is approved for the treatment of Pompe's Disease, an autosomal recessive disorder with a broad clinical spectrum. A Myozyme™ moiety is produced by a CHO cell line and possesses an amino acid sequence that is identical to the naturally occurring form of the enzyme.

The enzymatic activity of an acid alpha-glucosidase moiety or a corresponding conjugate thereof can be determined, e.g., using a 4-methylumbelliferyl-α-D-glucoside (Sigma) as an artificial substrate (Zwerschke et al. (2000) J. Biol. Chem. 275(13):9534-9541; Lu et al. (2003) Gene Therapy 10:1910-1916).

A water soluble polymer conjugate of an acid alpha-glucosidase moiety is useful for treating any condition responsive to treatment with acid alpha-glucosidase. For instance, such conjugates may by used to treat Pompe's Disease. Patients suffering from Pompe's Disease lack production of the naturally occurring enzyme. This lysosomal enzyme deficiency causes glycogen to accumulate in cardiac, respiratory, and skeletal muscle tissues, leading to the development of cardiomyopathy and progressive muscle weakness, including impairment of respiratory function. Patients with infantile-onset Pompe's Disease experience a progressively deteriorating illness usually leading to death within 1-2 years from the time of diagnosis.

Iduronate-2-sulfatase (e.g., Idursulfase or Elaprase)

Another exemplary and preferred lysosomal enzyme moiety is an iduronate-2-sulfatase moiety.

Iduronate-2-sulfatase (“IDS”; NCBI GenBank Accession No. NP_{—}000193; also see Entrez GeneID No. 3423) acts as an exosulfatase in lysosomes to hydrolyze the C2-sulfate ester bond from non-reducing-terminal iduronic acid residues in the glycosaminoglycans heparan sulfate and dermatan sulfate. IDS is one of a family of at least nine sulfatases that hydrolyze sulfate esters in human cells. They are all lysosomal enzymes that act on sulfated monosaccharide residues in a variety of complex substrates with the exception of microsomal steroid sulfatase (or arylsulfatase C), which acts on sulfated 3β-hydroxysteriods (1,2). Each sulfatase displays absolute substrate specificity. A deficiency in the activity of IDS in humans leads to the lysosomal accumulation of heparan sulfate and dermatan sulfate fragments and their excretion in urine. This storage results in the clinical disorder, Hunter syndrome (mucopolysaccharidosis type II, MPS-II), in which patients may present with variable phenotypes from severe mental retardation, skeletal deformities, and stiff joints to a relatively mild course.

In one embodiment, an iduronate-2-sulfatase moiety will possess the amino acid sequence corresponding to NCBI GenBank Accession No. NP_{—}000193.

Alternatively, an iduronate-2-sulfatase moiety will possess a sequence corresponding to that of Elaprase™ (idursulfase). Elaprase™ is a purified recombinant form of iduronate-2-sulfatase marketed by Shire Pharmaceuticals (Cambridge, Mass.) for treatment of MPS-II. As recombinantly prepared, idursulfase is expressed as a monomeric protein of 550-amino acid glycoprotein; the glycoprotein is secreted into the medium as a mature protein of 525 amino acids with a molecular weight of approximately 76 kilodaltons following cleavage of the 25 amino acid signal peptide. Elaprase™ contains two disulfide bonds and eight asparagine-linked glycosylation sites occupied by complex oligosaccharide structures. The presence of mannose-6-phosphate (M6P) residues allows specific binding to M6P receptors on the cell surface, leading to cellular internalization and targeting to lysosomes. The enzyme activity of idursulfase is dependent on the post-translational modification of a specific cysteine at position 59 to formylglycine.

Glycosylation variants also suitable for use as an IDS moiety are described in U.S. Pat. Nos. 6,153,188 and 6,541,254.

Enzymatic activity of an iduronate-2-sulfatase moiety or its corresponding conjugates can be assessed using techniques known in the art, e.g., see Braun et al. (1993) Proc. Natl. Acad. Sci. USA, 90:11830-11834. Preferred conjugates are those possessing some degree of iduronate-2-sulfatase moiety activity, although such activity is not essential for conjugates having cleavable linkages.

A water-soluble polymer conjugate of iduronate-2-sulfatase may be used, for example, in treating Hunter syndrome (Mucopolysaccharidosis II, or MPS II), a rare inherited disease which can lead to premature death. Hunter Syndrome usually becomes apparent in children one to three years of age, and its symptoms include growth delay, joint stiffness, and coarsening of facial features. In severe cases, patients can experience respiratory and cardiac problems, enlargement of the liver and spleen, neurological deficits, and even death.

Additional lysosomal enzymes suitable for use in the conjugates provided herein include those described in Table 1, among others.

The Water-Soluble Polymer

As previously discussed, each conjugate comprises a lysosomal enzyme moiety covalently attached to a water-soluble polymer. With respect to the water-soluble polymer, the water-soluble polymer is non-peptidic, nontoxic, non-naturally occurring and biocompatible. With respect to biocompatibility, a substance is considered biocompatible if the beneficial effects associated with use of the substance alone or with another substance (e.g., an active agent such as an glucocerebrosidase moiety) in connection with living tissues (e.g., administration to a patient) outweighs any deleterious effects as evaluated by a clinician, e.g., a physician. With respect to non-immunogenicity, a substance is considered non-immunogenic if the intended use of the substance in vivo does not produce an undesired immune response (e.g., the formation of antibodies) or, if an immune response is produced, that such a response is not deemed clinically significant or important as evaluated by a clinician. It is particularly preferred that the non-peptidic water-soluble polymer and its corresponding conjugate is biocompatible and non-immunogenic.

Further, the polymer itself is typically characterized as having from 2 to about 300 termini (prior to attachment to a lysosomal enzyme moiety). Examples of such polymers include, but are not limited to, poly(alkylene glycols) such as polyethylene glycol (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and combinations of any of the foregoing.

Polyoxazolines for use in the conjugates described herein include activated polyoxazolines such as described in International Patent Publication No. WO 2008/106186.

The water soluble polymer is not limited to a particular structure and can be linear (e.g., an end capped, e.g., alkoxy PEG or a bifunctional PEG), a branched or multi-armed PEG (e.g., forked PEG or PEG attached to a polyol core), a dendritic PEG, or star PEG, or any of the foregoing further comprising one or more degradable linkages. Moreover, the internal structure of the water-soluble polymer can be organized in any number of different repeat patterns and can be selected from the group consisting of homopolymer, alternating copolymer, random copolymer, block copolymer, alternating tripolymer, random tripolymer, and block tripolymer.

Typically, activated PEG and other activated water-soluble polymers (i.e., polymeric reagents) are activated with a suitable activating group appropriate for coupling to a desired site on the lysosomal enzyme moiety, e.g., a glucocerebrosidase moiety. Thus, a polymeric reagent will possess a reactive group for reaction with the lysosomal enzyme moiety. Representative polymeric reagents and methods for conjugating these polymers to an active moiety are known in the art and further described in Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182. Exemplary activating groups suitable for coupling to a lysosomal enzyme moiety include hydroxyl, maleimide, ester and preferably activated ester, acetal, ketal, amine, carboxyl, aldehyde, aldehyde hydrate, ketone, vinyl ketone, thione, thiol, vinyl sulfone, hydrazine, among others.

For example, PEG-diol or methoxy-PEG-OH can be purchased from any of a number of commercial suppliers such as VWR and then further functionalized to contain one or more desired reactive groups. Low molecular weight PEG reagents (typically containing from 2 to about 24 monomer subunits) are available from ThermoScientific (Pierce Protein Research Products). Exemplary reagents available from ThermoScientific include methoxy-PEG-NHS (N-hydroxysuccinimidyl ester), TMS-PEG, a tri-branched PEG having a single attachment site for covalent attachment to a lysosomal storage enzyme, MM(PEG), a methoxy PEG reagent having a maleimide terminus, among other reagents provided in the online catalog, 2008, under “PEGylation Reagents”. Additional sources for PEG reagents having a wide variety of molecular weights, geometries, and functionalities include JenKem Technology USA. See, e.g., JenKem Technology USA Product List, 2008, incorporated herein by reference. Available reagents include linear, Y-shaped, and multi-armed reactive PEG polymers. Additional suppliers of suitable PEG reagents include but are not limited to, IRIS Biotech GmbH, NOF Corporation, and Laysan Bio, the 2008 product listings of which are herein incorporated by reference.

Typically, the weight-average molecular weight of the water-soluble polymer in the conjugate is from about 100 Daltons to about 150,000 Daltons. Exemplary ranges, however, include weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. For any given water-soluble polymer, PEGs having a molecular weight in one or more of these ranges are preferred.

Exemplary weight-average molecular weights for the water-soluble polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the water-soluble polymer (e.g., a branched 40,000 Dalton water-soluble polymer comprised of two 20,000 Dalton polymers) having a total molecular weight of any of the foregoing can also be used. In one or more embodiments, the conjugate will not have any PEG moieties attached, either directly or indirectly, with a PEG having a weight average molecular weight of less than about 6,000 Daltons.

When used as the polymer, PEGs will typically comprise a number of (OCH₂CH₂) monomers (or (CH₂CH₂O) monomers, depending on how the PEG is defined). As used throughout the description, the number of repeating units is identified by the subscript “n” in “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

One particularly preferred polymer for use in the invention is an end-capped polymer, that is, a polymer having at least one terminus capped with a relatively inert group, such as a lower C₁₋₆ alkoxy group, although a hydroxyl group can also be used. When the polymer is PEG, for example, it is preferred to use a methoxy-PEG (commonly referred to as mPEG), which is a linear form of PEG wherein one terminus of the polymer is a methoxy (—OCH₃) group, while the other terminus is a hydroxyl or other functional group that can be optionally chemically modified.

In one form useful in one or more embodiments of the present invention, free or unbound PEG is a linear polymer terminated at each end with hydroxyl groups:

HO—CH₂CH₂O—(CH₂CH₂O)ₙ—CH₂CH₂—OH,

wherein (n) typically ranges from zero to about 4,000.

The above polymer, alpha-, omega-dihydroxylpoly(ethylene glycol), can be represented in brief form as HO-PEG-OH where it is understood that the -PEG- symbol can represent the following structural unit:

—CH₂CH₂O—(CH₂CH₂O)ₙ—CH₂CH₂—,

wherein (n) is as defined as above.

Another type of PEG useful in one or more embodiments of the present invention is methoxy-PEG-OH, or mPEG in brief, in which one terminus is the relatively inert methoxy group, while the other terminus is a hydroxyl group. The structure of mPEG is given below.

CH₃O—CH₂CH₂O—(CH₂CH₂O)ₙ—CH₂CH₂—OH

wherein (n) is as described above.

Multi-armed or branched PEG molecules, such as those described in U.S. Pat. No. 5,932,462, can also be used as the PEG polymer portion of the lysosomal enzyme conjugate. For example, PEG can have the structure:

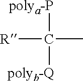

wherein:

polyₐ and poly_{b} are PEG backbones (either the same or different), such as methoxy poly(ethylene glycol);

R″ is a nonreactive moiety, such as H, methyl or a PEG backbone; and

P and Q are nonreactive linkages. In a preferred embodiment, the branched PEG polymer is methoxy poly(ethylene glycol) disubstituted lysine. Depending on the specific lysosomal enzyme moiety, e.g., glucocerebrosidase moiety, used, the reactive ester functional group of the disubstituted lysine may be further modified to form a functional group suitable for reaction with the target group within the lysosomal enzyme moiety.

In addition, the PEG can comprise a forked PEG. An example of a forked PEG is represented by the following structure:

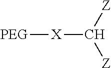

wherein X is a spacer moiety of one or more atoms and each Z is an activated terminal group linked to CH by a chain of atoms of defined length. International Application No. PCT/US99/05333, discloses various forked PEG structures capable of use in one or more embodiments of the present invention. The chain of atoms linking the Z functional groups to the branching carbon atom serve as a tethering group and may comprise, for example, alkyl chains, ether chains, ester chains, amide chains and combinations thereof.

The PEG polymer may comprise a pendant PEG molecule having reactive groups, such as carboxyl, covalently attached along the length of the PEG rather than at the end of the PEG chain. The pendant reactive groups can be attached to the PEG directly or through a spacer moiety, such as an alkylene group.

In addition to the above-described forms of PEG, the polymer can also be prepared with one or more weak or degradable linkages in the polymer, including any of the above-described polymers. For example, PEG can be prepared with ester linkages in the polymer that are subject to hydrolysis. As shown below, this hydrolysis results in cleavage of the polymer into fragments of lower molecular weight:

-PEG-CO₂-PEG-+H₂O→-PEG-CO₂H+HO-PEG-

Other hydrolytically degradable linkages, useful as a degradable linkage within a polymer backbone or as degradable linkage to a lysosomal enzyme moiety, include carbonate, imine, phosphate ester, hydrazone, acetal, orthoester, amide, carboxyl, urethane, peptide, oligonucleotide linkages formed by, for example, a phosphoramidite group, e.g., at the end of a polymer, and a 5′ hydroxyl group of an oligonucleotide, among others.

Such optional features of the conjugate, i.e., the introduction of one or more degradable linkages into the polymer chain or to the lysosomal enzyme moiety itself, may provide for additional control over the final desired pharmacological properties of the conjugate upon administration. For example, a conjugate exhibiting very little or no lysosomal enzyme activity (e.g., having one or more high molecular weight PEG chains attached thereto, for example, one or more PEG chains having a molecular weight greater than about 10,000, or wherein the conjugate is unable to target the lysosome or bind to its intended substrate) may be designed such that subsequent to administration, the conjugate is hydrolyzed to generate a bioactive conjugate possessing a portion of the original PEG chain, or from which the PEG chain is released all together. In this way, the properties of the conjugate can be more effectively tailored to provide the necessary targeting to the lysosome as well as bioactivity.

As described above, the water-soluble polymer, when attached to the lysosomal enzyme moiety, can also be “releasable.” That is, the water-soluble polymer cleaves (either through hydrolysis, enzymatic processes, or otherwise), thereby resulting in the unconjugated lysosomal enzyme moiety such as a glucocerebrosidase moiety. In some instances, cleavable polymers detach from the glucocerebrosidase moiety in vivo without leaving any fragment of the water-soluble polymer. In other instances, cleavable polymers detach from the glucocerebrosidase moiety in vivo leaving a relatively small fragment (e.g., a succinate tag) from the water-soluble polymer. An exemplary cleavable polymer includes one that attaches to the glucocerebrosidase moiety or any other lysosomal enzyme moiety via a carbonate linkage. In one embodiment, cleavage occurs under conditions such as those found in the lysosomal compartment, e.g., at pHs ranging from about 4.5-5.5.

Those of ordinary skill in the art will recognize that the foregoing discussion concerning non-peptidic and water-soluble polymers is by no means exhaustive and is merely illustrative, and that all polymeric materials having the qualities described above are contemplated. As used herein, the term “polymeric reagent” generally refers to an entire molecule, which can comprise a water-soluble polymer segment and a functional group.

Conjugate of a Lysosomal Enzyme Moiety

As described above, a conjugate as provided herein comprises a water-soluble polymer covalently attached to a lysosomal enzyme moiety. In a preferred embodiment, the lysosomal enzyme moiety is a glucocerebrosidase moiety. Typically, for any given conjugate, there will be one to three water-soluble polymers covalently attached to one or more moieties having lysosomal enzyme activity. In some instances, however, the conjugate may have 1, 2, 3, 4, 5, 6, 7, 8 or more water-soluble polymers individually attached to a lysosomal enzyme moiety. The water soluble polymer may be covalently attached to either an amino acid or to a carbohydrate portion of the glycoprotein, i.e., lysosomal enzyme moiety. Targeted carbohydrate modification may be carried out, e.g., using metabolic functionalization employing sialic acid-azide chemistry (Luchansky et al. (2004) Biochemistry 43(38), 12358) or other suitable approaches such as the use of glycidol to facilitate the introduction of aldehyde groups (Heldt et al. (2007) European Journal of Organic Chemistry 32:5429-5433).

The particular linkage within the moiety having lysosomal enzyme activity and the polymer depends on a number of factors. Such factors include, for example, the particular linkage chemistry employed, the particular lysosomal enzyme moiety, the available functional groups within the lysosomal enzyme moiety (either for attachment to a polymer or conversion to a suitable attachment site), the presence of additional reactive functional groups or carbohydrate moieties within the lysosomal enzyme moiety, and the like.

The conjugates of the invention can be, although are not necessarily, prodrugs, meaning that the linkage between the polymer and the lysosomal enzyme moiety is hydrolytically degradable to allow release of the parent moiety. Exemplary degradable linkages include carboxylate ester, phosphate ester, thiolester, anhydrides, acetals, ketals, acyloxyalkyl ether, imines, orthoesters, peptides and oligonucleotides. Such linkages can be readily prepared by appropriate modification of either the lysosomal enzyme moiety (e.g., the carboxyl group C terminus of the protein or a side chain hydroxyl group of an amino acid such as serine or threonine contained within the protein, or a similar functionality within the carbohydrate) and/or the polymeric reagent using coupling methods commonly employed in the art. Most preferred, however, are hydrolyzable linkages that are readily formed by reaction of a suitably activated polymer with a non-modified functional group contained within the moiety having lysosomal enzyme activity.

Alternatively, a hydrolytically stable linkage, such as an amide, urethane (also known as carbamate), amine, thioether (also known as sulfide), or urea (also known as carbamide) linkage can also be employed as the linkage for coupling the lysosomal enzyme moiety. Again, a preferred hydrolytically stable linkage is an amide. In one approach, a water-soluble polymer bearing an activated ester can be reacted with an amine group on the lysosomal enzyme moiety to thereby result in an amide linkage.

As described above, the conjugates (as opposed to an unconjugated lysosomal enzyme moiety) may or may not possess a measurable degree of lysosomal enzyme activity. For example, a polymer-glucocerebrosidase moiety conjugate in accordance with the invention will possesses anywhere from about 0.1% to about 100% of the bioactivity of the unmodified parent glucocerebrosidase moiety. In some instances, the polymer-glucocerebrosidase moiety conjugates may posses greater than 100% bioactivity of the unmodified parent glucocerebrosidase moiety. Preferably, conjugates possessing little or no glucocerebrosidase activity contain a hydrolyzable linkage connecting the polymer to the moiety, so that regardless of the lack (or relative lack) of activity in the conjugate, the active parent lysosomal enzyme molecule (or a derivative thereof) is released upon aqueous-induced cleavage of the hydrolyzable linkage. Such activity may be determined using a suitable in-vivo or in-vitro model such as described above, depending upon the known activity of the particular lysosomal enzyme moiety employed.

For conjugates possessing a hydrolytically stable linkage that couples the moiety having lysosomal enzyme activity to the polymer, the conjugate will typically possess a measurable degree of bioactivity. For instance, such conjugates are typically characterized as having an enzymatic activity satisfying one or more of the following percentages relative to that of the unconjugated lysosomal enzyme moiety: at least about 2%, at least about 5%, at least about 10%, at least about 15%, at least about 25%, at least about 30%, at least about 40%, at least about 50%, at least about 60%, at least about 80%, at least about 85%, at least about 90%, at least about 95%, at least about 97%, at least about 100%, and more than 105% (when measured in a suitable model, such as those well known in the art). Preferably, conjugates having a hydrolytically stable linkage (e.g., an amide linkage) will possess at least some degree of the lysosomal enzyme activity of the unmodified parent moiety.

Preferred conjugates as provided herein will also maintain their ability to target the related cell surface receptors on the lysosome; such targeting can be assessed by using a suitable assay designed to assess the lysosomal targeting capability of the subject lysosomal enzyme moiety or its related water-soluble polymer conjugate; the choice of water soluble polymer and positions of attachment will preferably be such that both the lysosomal targeting ability and enzymatic activity of the conjugate will be substantially maintained in comparison to that of the unmodified parent lysosomal enzyme. In one embodiment, a glycosylation independent targeting system such as that described in LeBowitz, J. H., et al., PNAS, 2004, 101 (9), 3083-3088, is utilized to deliver the subject conjugate to the lysosomes. In a preferred embodiment, the lysosomal enzyme polymer conjugate is pre-incubated with a slow-binding inhibitor such as isofagomine prior to administration to improve lysosomal delivery. Other slow-binding inhibitors may also be used. The conjugates provided herein may also be further modified or combined with an agent useful to promote targeting to the lysosomes, e.g., of the reticuloendothelial macrophages.

Exemplary conjugates in accordance with the invention will now be described. In a preferred embodiment, the lysosomal enzyme moiety is a glucocerebrosidase protein. Typically, such a protein is expected to share (at least in part) a similar amino acid sequence as the sequence provided in SEQ ID NO: 1. Thus, while reference will be made to specific locations or atoms within SEQ ID NO: 1, such a reference is for convenience only and one having ordinary skill in the art will be able to readily determine the corresponding location or atom in other moieties having glucocerebrosidase activity. In particular, the description provided herein for native human glucocerebrosidase is often applicable to fragments, deletion variants, substitution variants or addition variants of any of the foregoing, as is the case for any of the subject lysosomal enzyme moieties described herein.

Amino groups on glucocerebrosidase moieties provide a point of attachment between the glucocerebrosidase moiety and the water-soluble polymer. Using the amino acid sequence provided in SEQ ID NOs: 1 through 4, it is evident that there are 22 lysine residues, each having an ε-amino acid that may be available for conjugation. Thus, exemplary attachment points of such glucocerebrosidase moieties include attachment at the amine side chain associated with a lysine at any one of positions 7, 74, 77, 79, 106, 155, 157, 186, 194, 198, 215, 224, 293, 303, 321, 346, 408, 413, 425, 441, 466 and 473. Corresponding positions of SEQ ID NOs 5 and 6 can also be used. Further, the N-terminal amine of any protein can also serve as a point of attachment.

There are a number of examples of suitable polymeric reagents useful for forming covalent linkages with available amines of a glucocerebrosidase or other lysosomal enzyme moiety. Specific examples, along with the corresponding conjugate, are provided in Table 1, below. In the table, the variable (n) represents the number of repeating monomeric units and “—NH-(LE)” represents the residue of the lysosomal enzyme moiety, e.g., glucocerebrosidase moiety, following conjugation to the polymeric reagent. While each polymeric portion [e.g., (OCH₂CH₂)ₙ or (CH₂CH₂O)ₙ] presented in Table 2 terminates in a “CH₃” group, other groups (such as H, alkyl and benzyl) can be substituted for the methyl.

TABLE 2
Amine-Specific Polymeric Reagents and the Corresponding Lysosomal Enzyme Moiety Conjugate Formed
Polymeric Reagent

| | |
|---|---|
| 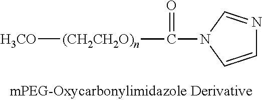 | 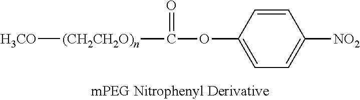 |
| 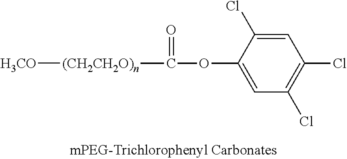 | 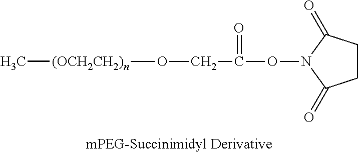 |

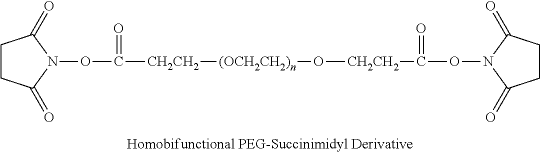

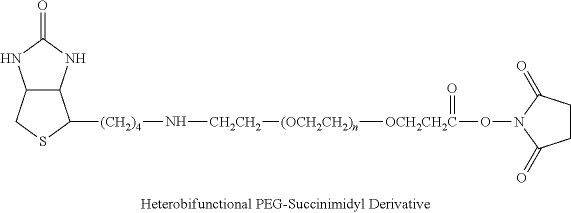

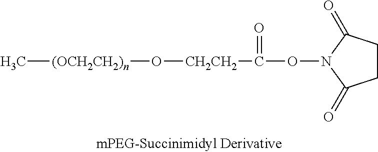

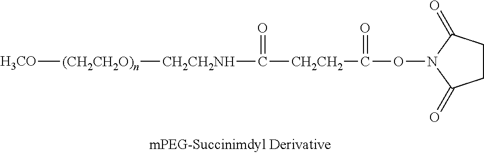

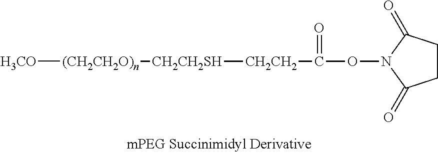

| | |
|---|---|
| 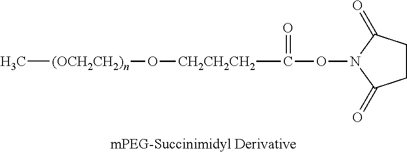 | 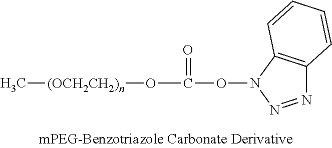 |

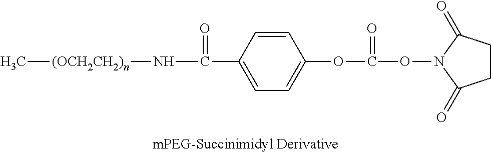

| | |
|---|---|
| 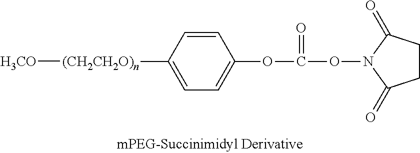 | 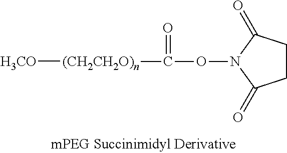 |

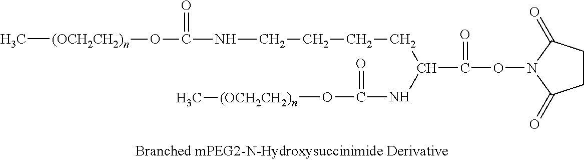

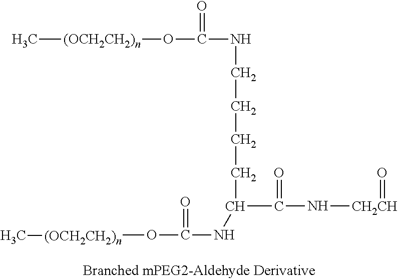

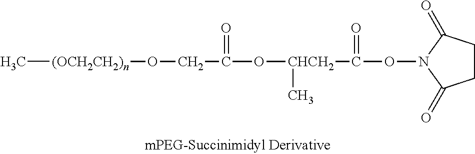

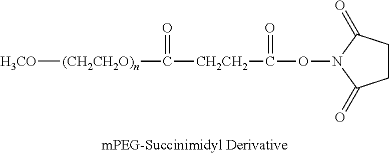

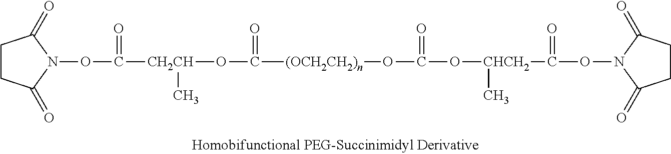

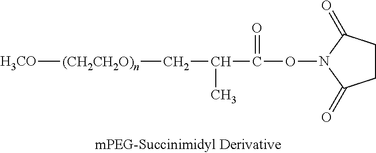

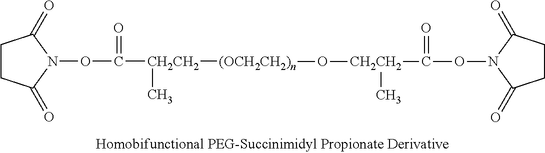

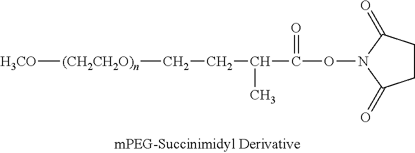

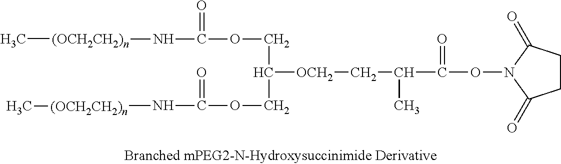

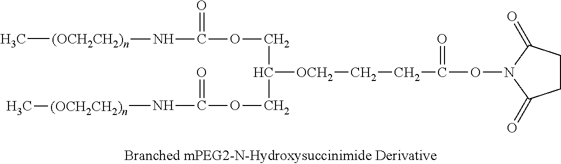

| | |
|---|---|
| 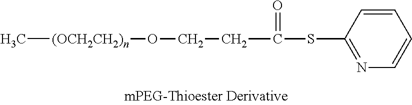 | 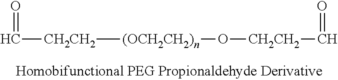 |
| 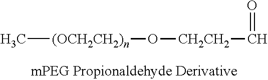 | 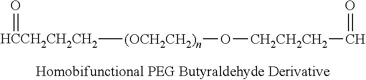 |
| 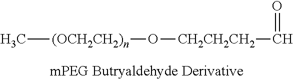 | 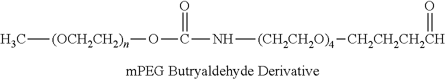 |

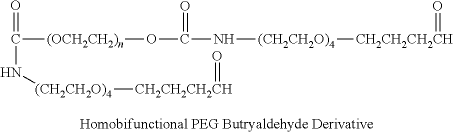

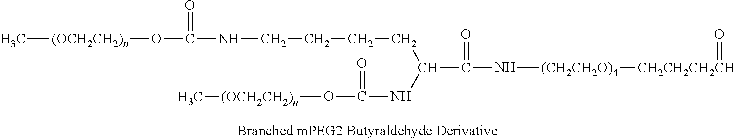

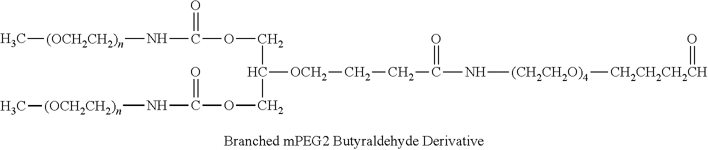

| | |
|---|---|
| 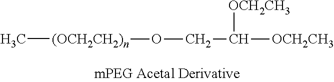 | 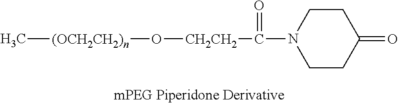 |
| 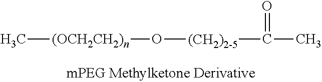 | 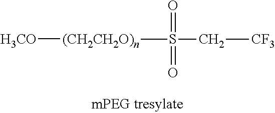 |
| 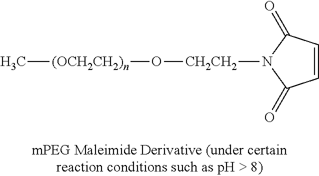 | 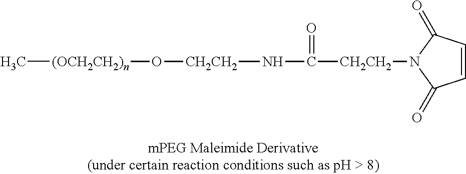 |

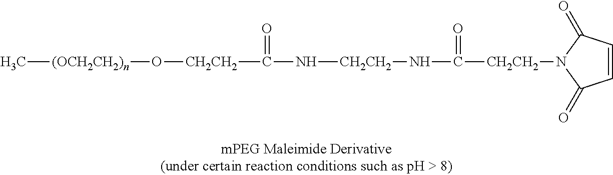

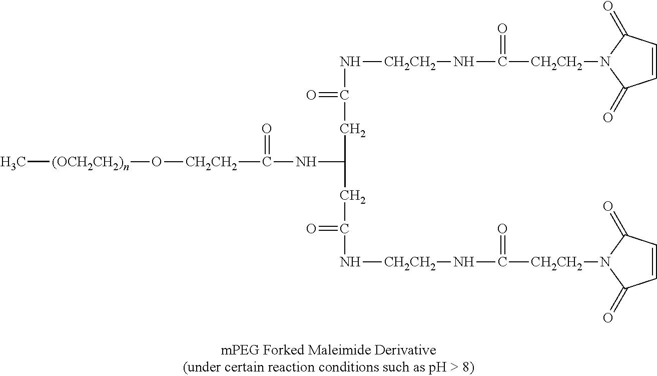

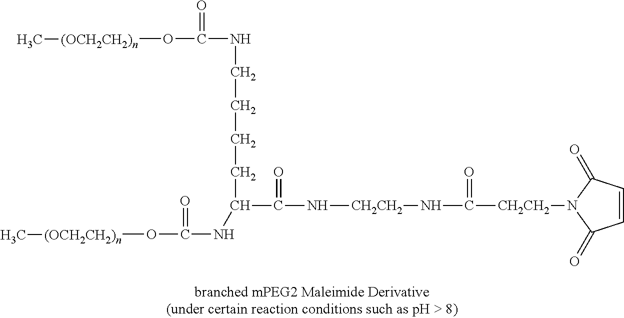

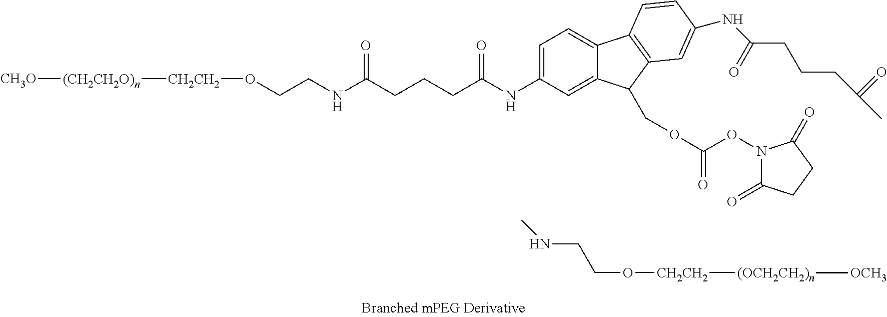

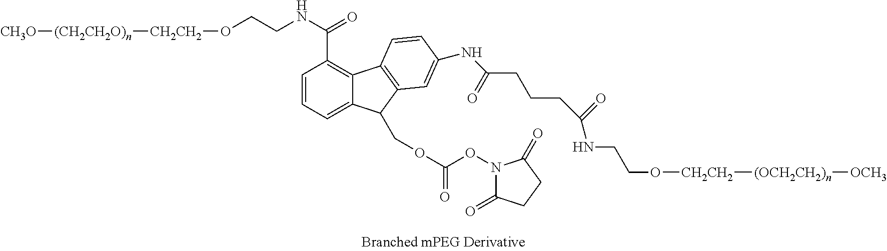

Corresponding Conjugate

| | |
|---|---|
| 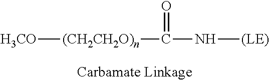 | 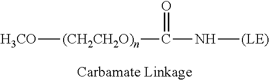 |
| 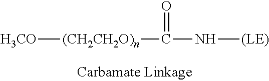 | 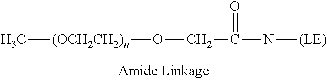 |

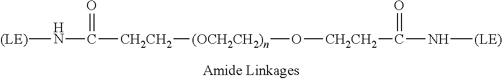

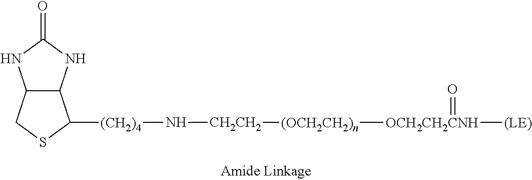

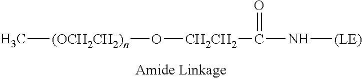

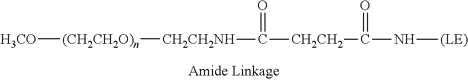

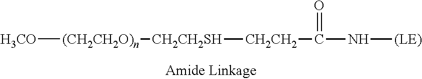

| | |
|---|---|
| 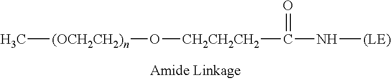 | 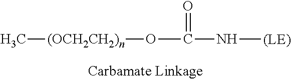 |

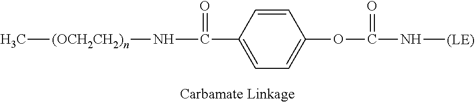

| | |
|---|---|
| 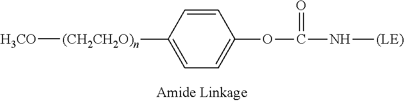 | 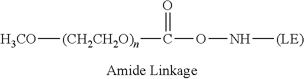 |

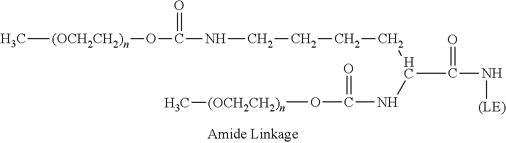

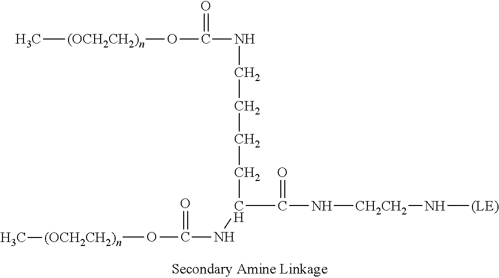

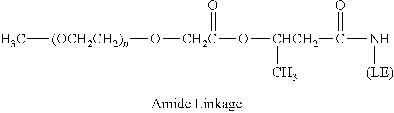

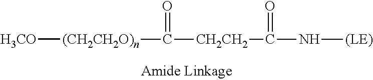

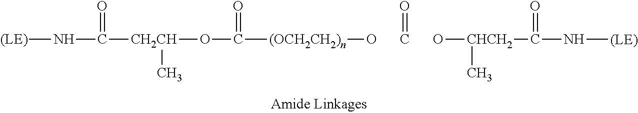

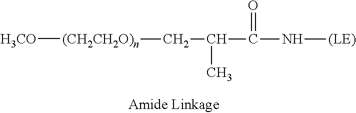

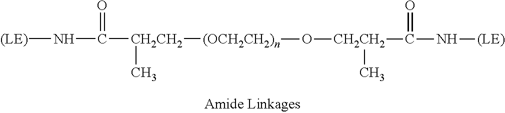

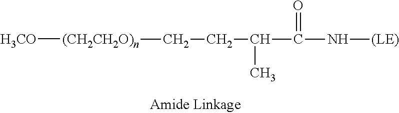

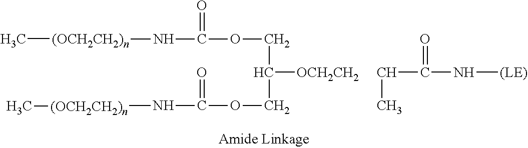

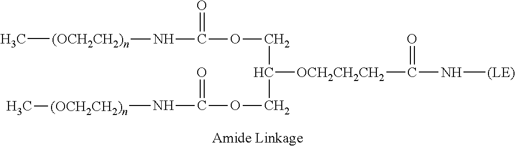

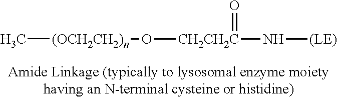

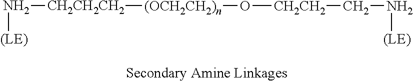

| | |
|---|---|
| H₃C—(OCH₂CH₂)ₙ—O—CH₂CH₂—CH₂—NH—(LE) Secondary Amine Linkage | 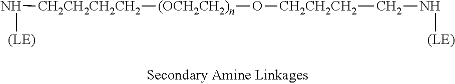 |

H₃C—(OCH₂CH₂)ₙ—O—CH₂CH₂CH₂—CH₂—NH—(LE)
Secondary Amine Linkage

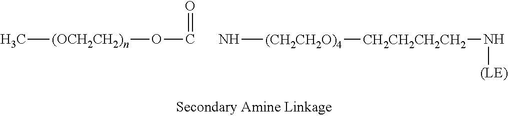

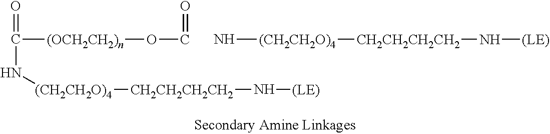

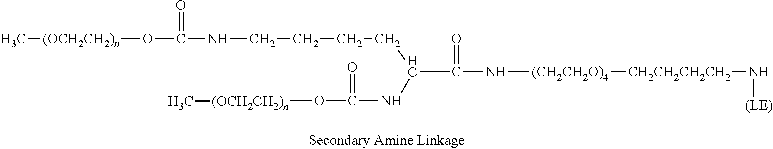

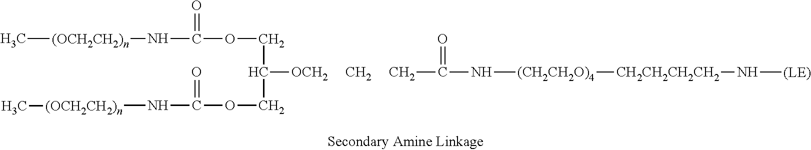

| | |
|---|---|
| H₃C—(OCH₂CH₂)ₙ—O—CH₂CH₂—NH—(LE) Secondary Amine Linkage | 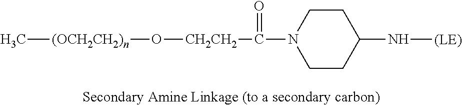 |
| 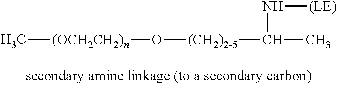 | H₃CO—(CH₂CH₂O)ₙ—CH₂CH₂—NH—(LE) Secondary Amine Linkage |

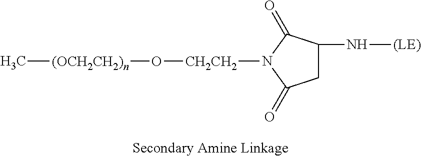

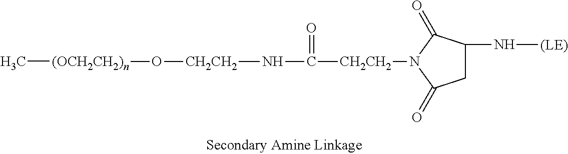

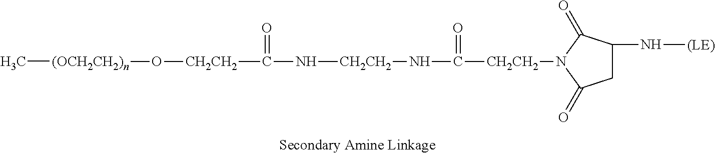

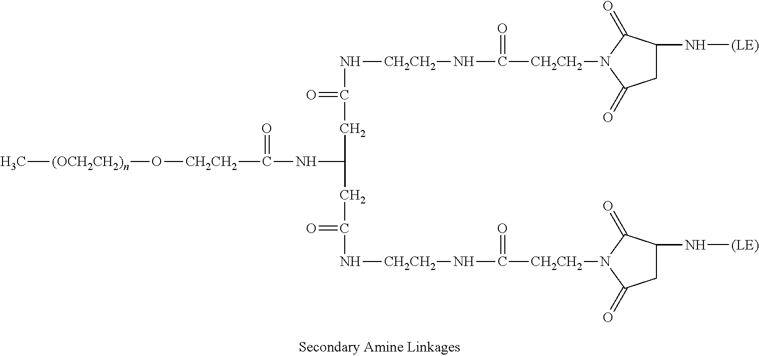

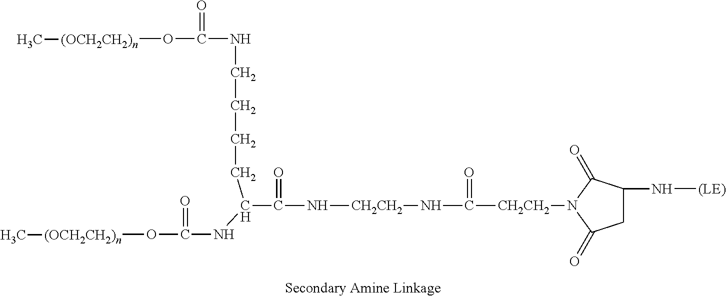

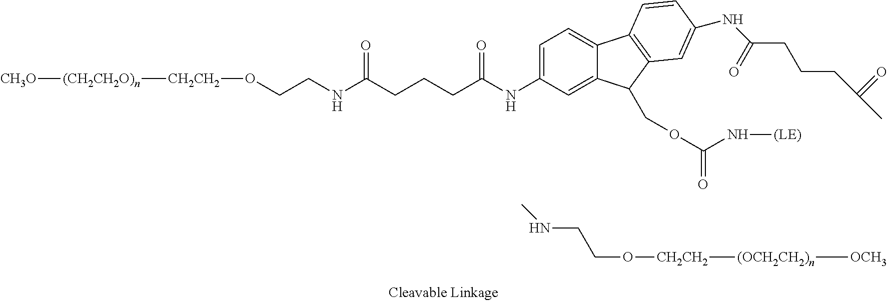

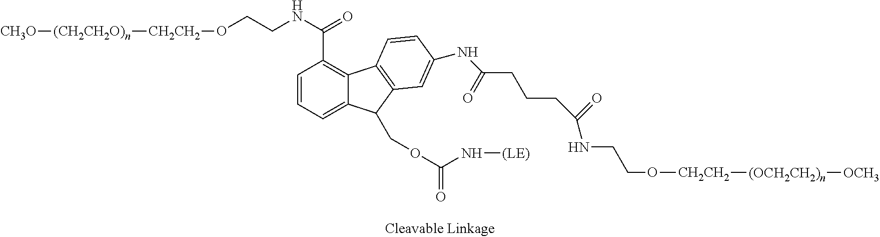

In several preferred embodiments of the structures in Table 2, the designation “LE” corresponds to a glucocerebrosidase moiety.

Conjugation of a polymeric reagent to an amino group of a lysosomal enzyme moiety such as a glucocerebrosidase moiety can be accomplished by a variety of techniques. In one approach, a lysosomal enzyme moiety can be conjugated to a polymeric reagent functionalized with a succinimidyl derivative (or other activated ester group, wherein approaches similar to those described for these alternative activated ester group-containing polymeric reagents can be used). In this approach, the polymer bearing a succinimidyl derivative can be attached to the lysosomal enzyme moiety in an aqueous medium at a pH of 7 to 9.0, although using different reaction conditions (e.g., a lower pH such as 6 to 7, or different temperatures and/or less than 15° C.) can result in the attachment of the polymer to a different location on the lysosomal enzyme moiety. In addition, an amide linkage can be formed reacting an amine-terminated non-peptidic, water-soluble polymer with a lysosomal enzyme moiety bearing an activated carboxylic acid group.

An exemplary conjugate comprises the following structure

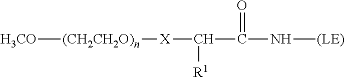

where (n) is an integer having a value of from 2 to 4000; X is a spacer moiety; R¹ is an organic radical (typically a lower alkyl group); and LE is a residue of a lysosomal enzyme moiety.

Another exemplary conjugate of the present invention comprises the following structure:

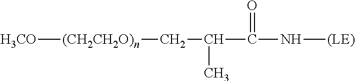

where (n) is an integer having a value of from 2 to 4000 and LE is a residue of a lysosomal enzyme moiety.

Typical of another approach useful for conjugating a lysosomal enzyme moiety to a polymeric reagent is use of reductive amination to conjugate a primary amine of a lysosomal enzyme moiety with a polymeric reagent functionalized with a ketone, aldehyde or a hydrated form thereof (e.g., ketone hydrate, aldehyde hydrate). In this approach, the primary amine from the lysosomal enzyme moiety reacts with the carbonyl group of the aldehyde or ketone (or the corresponding hydroxyl-containing group of a hydrated aldehyde or ketone), thereby forming a Schiff base. The Schiff base, in turn, can then be reductively converted to a stable conjugate through use of a reducing agent such as sodium borohydride. Selective reactions (e.g., at the N-terminus are possible) are possible, particularly with a polymer functionalized with a ketone or an alpha-methyl branched aldehyde and/or under specific reaction conditions (e.g., reduced pH).

Exemplary conjugates where the water-soluble polymer is in a branched form may comprises the branched form of the water-soluble polymer the following structure:

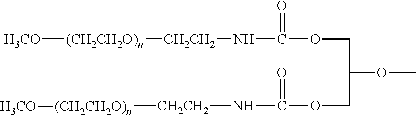

where each (n) is independently an integer having a value of from 2 to 4000. In a preferred embodiment, both (n) values are approximately the same, so that each arm is identical. Related exemplary conjugates may correspond to the following structure:

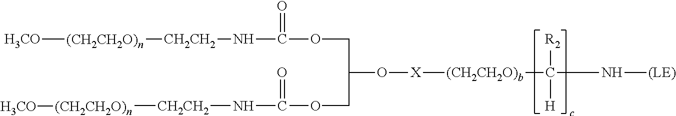

where each (n) is independently an integer having a value of from 2 to 4000; X is spacer moiety; (b) is an integer having a value 2 through 6; (c) is an integer having a value 2 through 6; R², in each occurrence, is independently H or lower alkyl; and LE is a residue of a lysosomal enzyme moiety.

Yet another exemplary conjugate may correspond to the following structure:

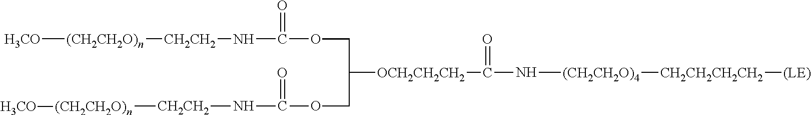

where each (n) is independently an integer having a value of from 2 to 4000; and LE is a residue of a lysosomal enzyme moiety.

A further exemplary conjugate corresponds to the following structure:

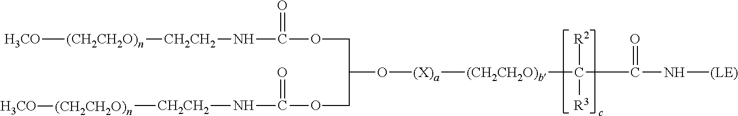

where each (n) is independently an integer having a value of from 2 to 4000; (a) is either zero or one; X, when present, is a spacer moiety comprised of one or more atoms; (b′) is zero or an integer having a value of one through ten; (c) is an integer having a value of one through ten; R², in each occurrence, is independently H or an organic radical; R³, in each occurrence, is independently H or an organic radical; and LE is a residue of a lysosomal enzyme moiety.

Additional exemplary conjugates may be characterized structurally as follows:

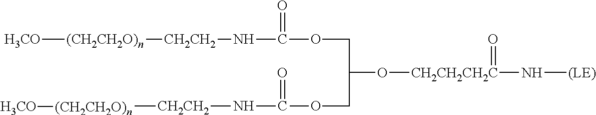

where each (n) is independently an integer having a value of from 2 to 4000; and LE is a residue of a lysosomal enzyme moiety.

Carboxyl groups represent another functional group that can serve as a point of attachment on the lysosomal enzyme moiety, e.g., a glucocerebrosidase moiety. The conjugate may be characterized generally as follows:

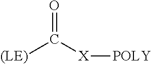

where (LE) and the adjacent carbonyl group corresponds to the carboxyl-containing lysosomal enzyme moiety, X is a linkage, preferably a heteroatom selected from O, N(H), and S, and POLY is a water-soluble polymer such as PEG, optionally terminating in an end-capping moiety.

The C(O)—X linkage results from the reaction between a polymeric derivative bearing a terminal functional group and a carboxyl-containing lysosomal enzyme moiety. As discussed above, the specific linkage will depend on the type of functional group utilized. If the polymer is end-functionalized or “activated” with a hydroxyl group, the resulting linkage will be a carboxylic acid ester and X will be O. If the polymer backbone is functionalized with a thiol group, the resulting linkage will be a thioester and X will be S. When certain multi-arm, branched or forked polymers are employed, the C(O)X moiety, and in particular the X moiety, may be relatively more complex and may include a longer linkage structure.

Water-soluble derivatives containing a hydrazide moiety are also useful for conjugation at a carbonyl. To the extent that the lysosomal enzyme moiety such as a glucocerebrosidase moiety does not contain a carbonyl moiety, a carbonyl moiety can be introduced by reducing any carboxylic acids (e.g., the C-terminal carboxylic acid) and/or by providing glycosylated or glycated (wherein the added sugars have a carbonyl moiety) versions of the lysosomal enzyme moiety. Specific examples of water-soluble derivatives containing a hydrazide moiety, along with the corresponding conjugates, are provided in Table 3, below. In addition, any water-soluble derivative containing an activated ester (e.g., a succinimidyl group) can be converted to contain a hydrazide moiety by reacting the water-soluble polymer derivative containing the activated ester with hydrazine (NH₂—NH₂) or tert-butyl carbazate [NH₂NHCO₂C(CH₃)₃]. In the table, the variable (n) represents the number of repeating monomeric units and “≡C-(LE)” represents the residue of the lysosomal enzyme moiety following conjugation to the polymeric reagent. Optionally, the hydrazone linkage can be reduced using a suitable reducing agent. While each polymeric portion [e.g., (OCH₂CH₂)ₙ or (CH₂CH₂O)ₙ] presented in Table 3 terminates in a “CH₃” group, other groups (such as H and benzyl) can be substituted for the illustrative methyl group.

TABLE 3
Carboxyl-Specific Polymeric Reagents and the Corresponding Lysosomal Enzyme Moiety Conjugate
Polymeric Reagent

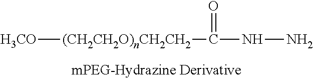

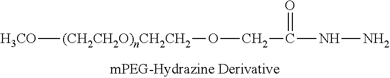

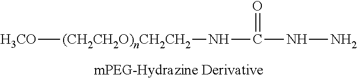

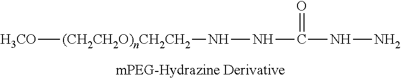

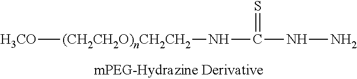

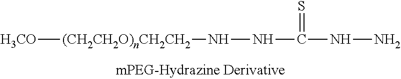

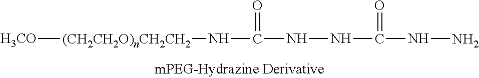

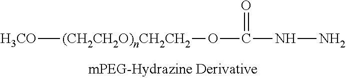

Corresponding Conjugate

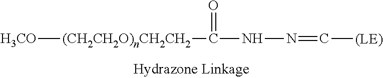

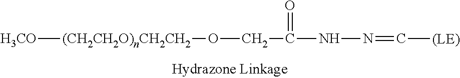

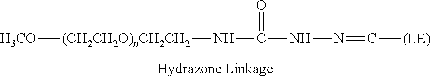

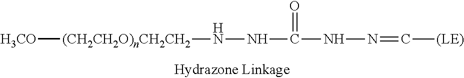

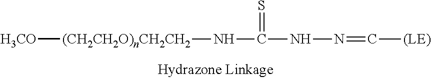

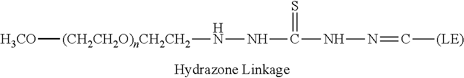

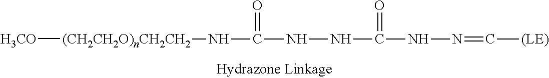

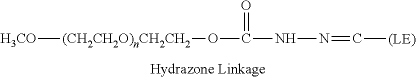

Thiol groups contained within the lysosomal enzyme moiety can serve as effective sites of attachment for the water-soluble polymer. In particular, cysteine residues provide thiol groups when the lysosomal enzyme moiety is a protein. The thiol groups in such cysteine residues can then be reacted with an activated PEG that is specific for reaction with thiol groups, e.g., an N-maleimidyl polymer or other derivative, as described in U.S. Pat. No. 5,739,208 and in International Patent Publication No. WO 01/62827. Alternatively, a protected thiol may be incorporated into an oligosaccharide side chain of an activated glycoprotein, followed by deprotection prior to reaction with a thiol-reactive water soluble polymer.

With respect to SEQ ID NOs: 1 through 4 corresponding to glucocerebrosidase moieties, there are seven thiol-containing cysteine residues. Thus, preferred thiol attachment sites are associated with one of these seven cysteine residues. Although it is preferred not to disrupt any disulfide bonds, it may be possible to attach a polymer within the side chain of one or more of these cysteine residues and retain a degree of activity. In addition, it is possible to add a cysteine residue to the lysosomal enzyme moiety using conventional synthetic techniques. See, for example, the procedure described in WO 90/12874 for adding cysteine residues, wherein such procedure can be adapted for a lysosomal enzyme moiety such as a glucocerebrosidase moiety. In addition, conventional genetic engineering processes can also be used to introduce a cysteine residue into the lysosomal enzyme moiety. In some embodiments, however, it is preferred not to introduce and additional cysteine residue and/or thiol group.

Specific examples, along with the resulting conjugate, are provided in Table 4, below. In the table, the variable (n) represents the number of repeating monomeric units and “—S-(LE)” represents the lysosomoal enzyme moiety residue following conjugation to the water-soluble polymer. While each polymeric portion [e.g., (OCH₂CH₂)ₙ or (CH₂CH₂O)ₙ] presented in Table 4 terminates in a “CH₃” group, other groups (such as H and benzyl) are also suitable. Also not always shown, the illustrative reagents and conjugates may also be prepared as symmetrical “dumbbell” type structures, such that the illustrative terminal methyl group is replaced with the identical portion shown to the right of the PEG monomer repeat units in the table and having a LE moiety attached at each terminus (e.g., by flipping the portion of the structure to the right of the (OCH₂CH₂)ₙ and replacing the terminal methyl therewith.

TABLE 4
Thiol-Specific Polymeric Reagents and the Corresponding Lysosomal Enzyme Moiety Conjugate
Polymeric Reagent

| | |
|---|---|
| 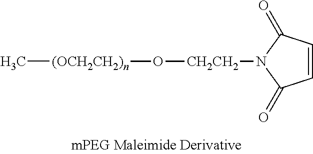 | 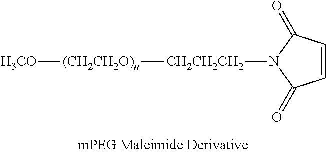 |

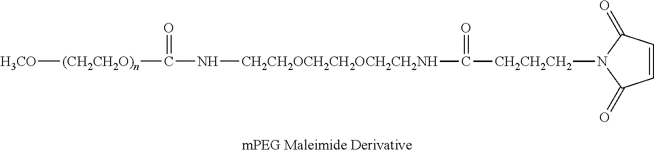

| | |
|---|---|
| 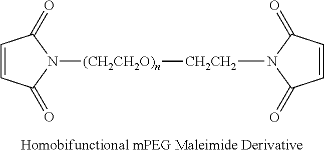 | 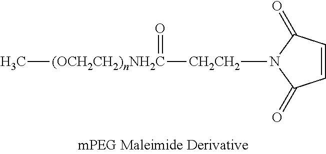 |

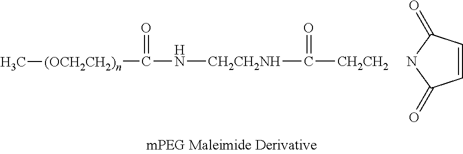

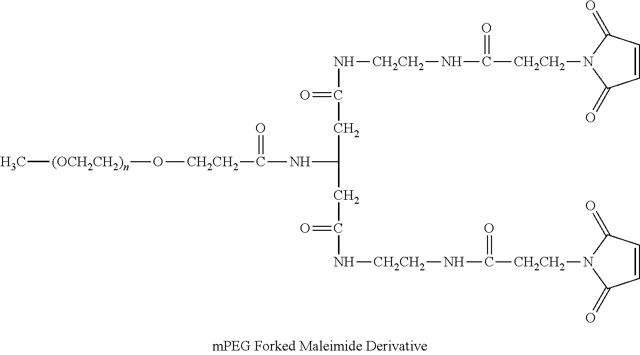

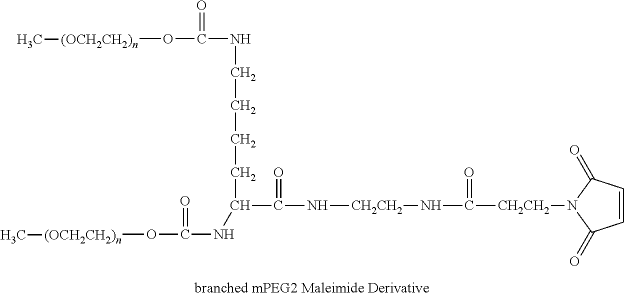

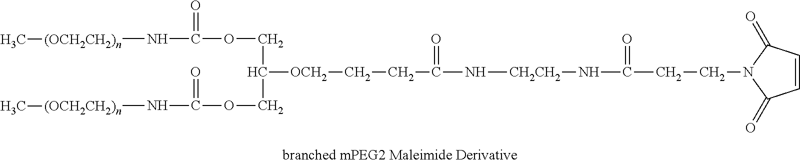

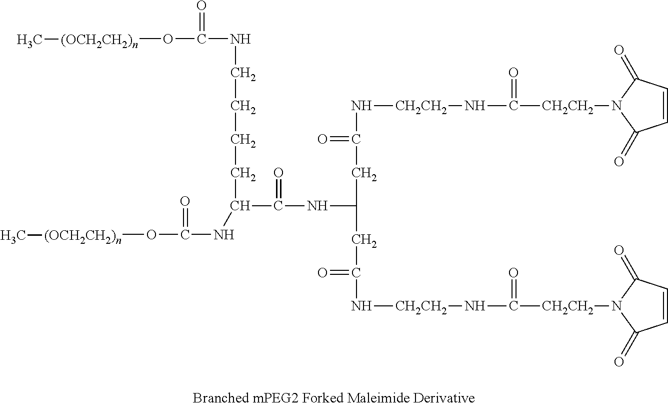

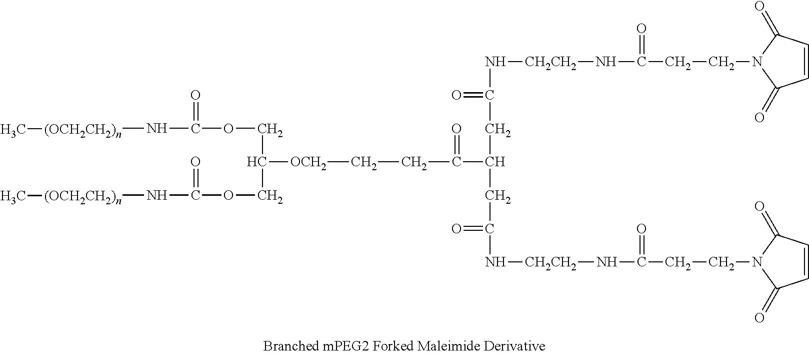

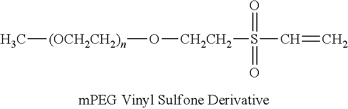

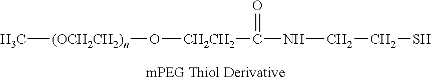

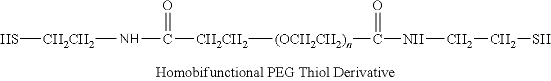

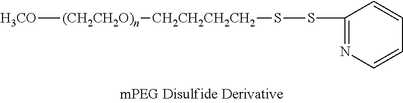

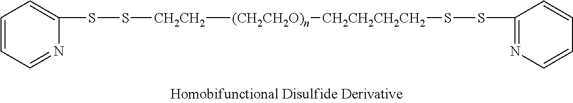

Corresponding Conjugate

| | |
|---|---|
| 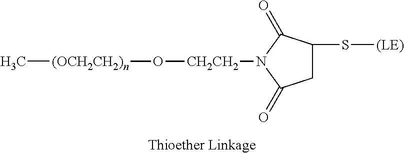 | 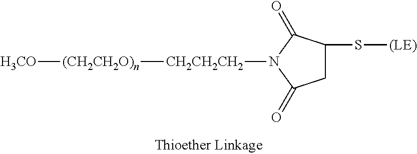 |

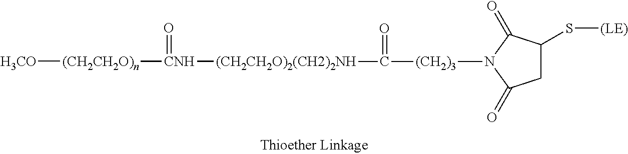

| | |
|---|---|
| 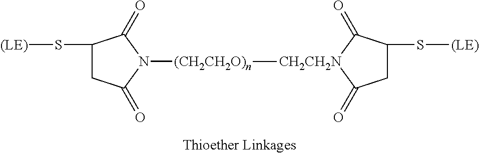 | 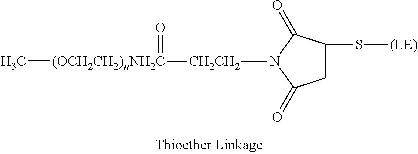 |

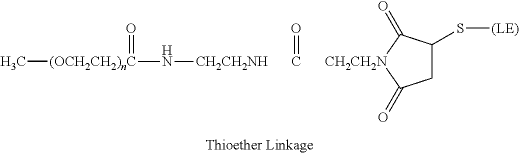

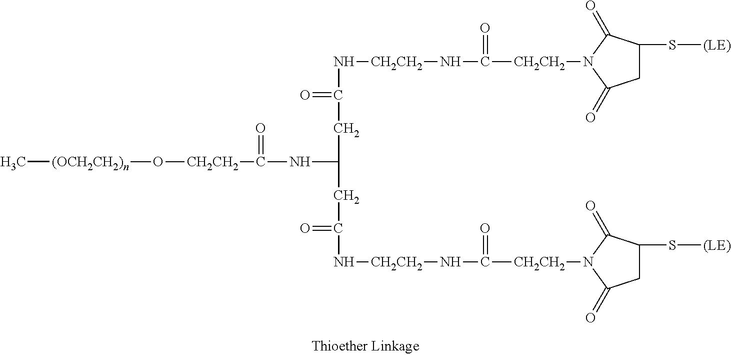

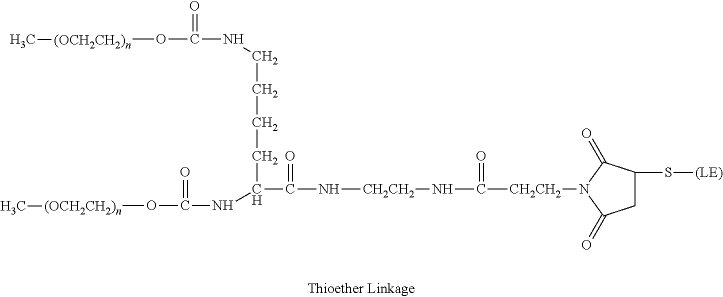

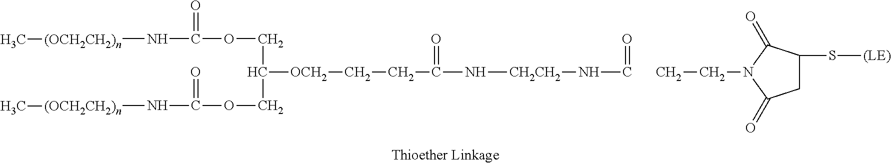

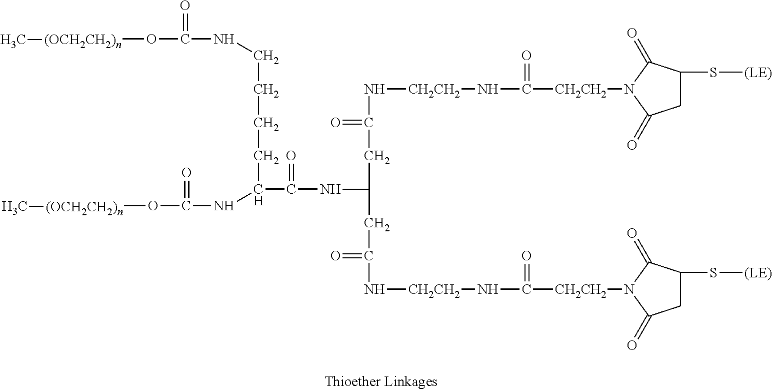

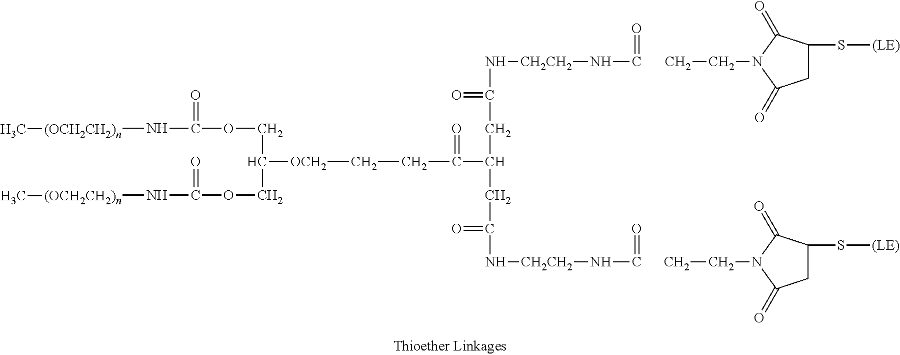

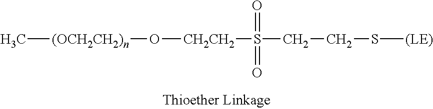

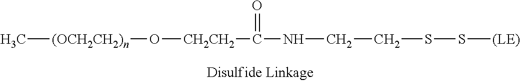

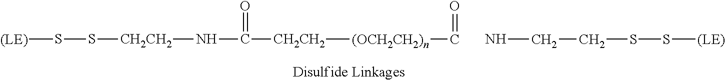

| | |
|---|---|
| H₃CO—(CH₂CH₂O)ₙ—CH₂CH₂CH₂CH₂—S S—(LE) | (LE)—S—S—CH₂CH₂—(CH₂CH₂O)ₙ—CH₂CH₂CH₂CH₂—S—S—(LE) |
| Disulfide Linkage | Disulfide Linkages |

With respect to conjugates formed from water-soluble polymers bearing one or more maleimide functional groups (regardless of whether the maleimide reacts with an amine or thiol group on the glucocerebrosidase moiety), the corresponding maleamic acid form(s) of the water-soluble polymer can also react with a lysosomal enzyme moiety such as a glucocerebrosidase moiety. Under certain conditions (e.g., a pH of about 7-9 and in the presence of water), the maleimide ring will “open” to form the corresponding maleamic acid. The maleamic acid, in turn, can react with an amine or thiol group of a lysosomal enzyme moiety. Exemplary maleamic acid-based reactions are schematically shown below. POLY represents the water-soluble polymer, and (LE) represents a lysosomal enzyme moiety.

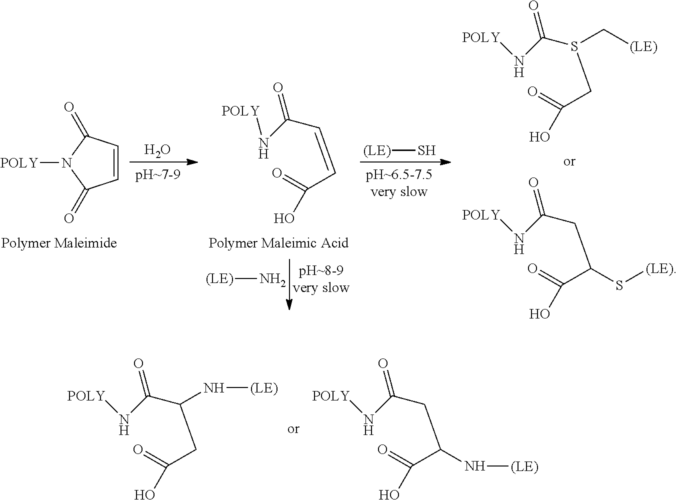

A representative conjugate may, e.g., have the following structure:

POLY-L_{0,1}-C(O)Z—Y—S—S-(LE)

wherein POLY is a water-soluble polymer, L is an optional linker, Z is a heteroatom selected from the group consisting of O, NH, and S, and Y is selected from the group consisting of C₂₋₁₀ alkyl, C₂₋₁₀ substituted alkyl, aryl, and substituted aryl, and (LE) is a lysosomal enzyme moiety. Polymeric reagents suitable for reaction with a lysosomal enzyme moiety and which form conjugates such as the foregoing are described in U.S. Patent Application Publication No. 2005/0014903.

Conjugates can be formed using thiol-specific polymeric reagents in a number of ways and the disclosure is not limited in this regard. For example, a glucocerebrosidase or other lysosomal enzyme moiety—optionally in a suitable buffer (including amine-containing buffers, if desired)—is placed in an aqueous media at a pH of about 7-8 and the thiol-specific polymeric reagent is added at a molar excess. The reaction is allowed to proceed for about 0.5 to 2 hours, although reaction times of greater than 2 hours (e.g., 5 hours, 10 hours, 12 hours, and 24 hours) can be useful if PEGylation yields are determined to be relatively low. Exemplary polymeric reagents that can be used in this approach are polymeric reagents bearing a reactive group selected from the group consisting of maleimide, sulfone (e.g., vinyl sulfone), and thiol (e.g., functionalized thiols such as an ortho pyridinyl or “OPSS”).

Preferred thiol groups in a lysosomal enzyme moiety such as a glucocerebrosidase moiety that can serve as a site for attaching a polymeric reagent include those thiol groups found within cysteine residues. Exemplary thiol groups associated with the side chain of the amino acid residue cysteine in SEQ ID NOs: 1 though 4 that can serve as an attachment point include positions 4, 16, 18, 23, 126, 248 and 342. Corresponding positions for SEQ ID NOs: 5 and 6 can also be used.

With respect to polymeric reagents, those described here and elsewhere can be purchased from various commercial sources or prepared from commercially available starting materials. In addition, methods for preparing polymeric reagents such as those described herein are described in the literature.

The attachment between the lysosomal enzyme moiety and the non-peptidic water-soluble polymer can be direct, wherein no intervening atoms are located between the lysosomal enzyme moiety and the polymer, or indirect, wherein one or more atoms are located between the lysosomal enzyme moiety and the polymer. With respect to the indirect attachment, a “spacer moiety” serves as a linker between the residue of the lysosomal enzyme moiety and the water-soluble polymer. The one or more atoms making up the spacer moiety can include one or more of carbon atoms, nitrogen atoms, sulfur atoms, oxygen atoms, and combinations thereof. The spacer moiety can comprise an amide, secondary amine, carbamate, thioether, and/or disulfide group. Nonlimiting examples of specific spacer moieties include those selected from the group consisting of —O—, —S—, —S—S—, —C(O)—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[CH₂]ₕ—(OCH₂CH₂)ⱼ—, bivalent cycloalkyl group, —O—, —S—, an amino acid, —N(R⁶)—, and combinations of two or more of any of the foregoing, wherein R⁶ is H or an organic radical selected from the group consisting of alkyl, substituted alkyl, alkenyl, substituted alkenyl, alkynyl, substituted alkynyl, aryl and substituted aryl, (h) is zero to six, and (j) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁₋₆ means that the structure can contain 1, 2, 3, 4, 5 or 6 methylenes. Additionally, any of the above spacer moieties may further include an ethylene oxide oligomer chain comprising 1 to 20 ethylene oxide monomer units [i.e., —(CH₂CH₂O)₁₋₂₀]. That is, the ethylene oxide oligomer chain can occur before or after the spacer moiety, and optionally in between any two atoms of a spacer moiety comprised of two or more atoms. Also, the oligomer chain would not be considered part of the spacer moiety if the oligomer is adjacent to a polymer segment and merely represent an extension of the polymer segment.

Compositions

The conjugates are typically part of a composition. Generally, the composition comprises a plurality of conjugates, preferably although not necessarily, each conjugate is comprised of the same lysosomal enzyme moiety (i.e., within the entire composition, only one type of lysosomal enzyme moiety is found). In addition, the composition can comprise a plurality of conjugates wherein any given conjugate is comprised of a moiety selected from the group consisting of two or more different lysosomal enzyme moieties (e.g., within the entire composition, two or more different glucocerebrosidase moieties are found). Optimally, however, substantially all conjugates in the composition (e.g., 85% or more of the plurality of conjugates in the composition) are each comprised of the same lysosomal enzyme moiety.

The composition can comprise a single conjugate species (e.g., a monoPEGylated conjugate wherein the single polymer is attached at the same location for substantially all conjugates in the composition) or a mixture of conjugate species (e.g., a mixture of monoPEGylated conjugates where attachment of the polymer occurs at different sites and/or a mixture monPEGylated, diPEGylated and triPEGylated conjugates). The compositions can also comprise other conjugates having four, five, six, seven, eight or more polymers attached to any given moiety having lysosomal enzyme activity. In addition, the invention includes instances wherein the composition comprises a plurality of conjugates, each conjugate comprising one water-soluble polymer covalently attached to one lysosomal enzyme moiety, as well as compositions comprising two, three, four, five, six, seven, eight, or more water-soluble polymers covalently attached to one lysosomal enzyme moiety.

With respect to the conjugates in the composition, the composition will satisfy one or more of the following characteristics: at least about 85% of the conjugates in the composition will have from one to four polymers attached to the lysosomal enzyme moiety; at least about 85% of the conjugates in the composition will have from one to four polymers attached to the lysosomal enzyme moiety; at least about 85% of the conjugates in the composition will have from one to three polymers attached to the lysosomal enzyme moiety; at least about 85% of the conjugates in the composition will have from one to two polymers attached to the lysosomal enzyme moiety; at least about 85% of the conjugates in the composition will have one polymer attached to the lysosomal enzyme moiety; at least about 95% of the conjugates in the composition will have from one to five polymers attached to the lysosomal enzyme moiety; at least about 95% of the conjugates in the composition will have from one to four polymers attached to the lysosomal enzyme moiety; at least about 95% of the conjugates in the composition will have from one to three polymers attached to the lysosomal enzyme moiety; at least about 95% of the conjugates in the composition will have from one to two polymers attached to the lysosomal enzyme moiety; at least about 95% of the conjugates in the composition will have one polymer attached to the lysosomal enzyme moiety; at least about 99% of the conjugates in the composition will have from one to five polymers attached to the lysosomal enzyme moiety; at least about 99% of the conjugates in the composition will have from one to four polymers attached to the lysosomal enzyme moiety; at least about 99% of the conjugates in the composition will have from one to three polymers attached to the lysosomal enzyme moiety; at least about 99% of the conjugates in the composition will have from one to two polymers attached to the lysosomal enzyme moiety; and at least about 99% of the conjugates in the composition will have one polymer attached to the lysosomal enzyme moiety.

In one or more embodiments, it is preferred that the conjugate-containing composition is free or substantially free of albumin. It is also preferred that the composition is free or substantially free of proteins that do not have lysosomal enzyme activity. Thus, it is preferred that the composition is 85%, more preferably 95%, and most preferably 99% free of albumin. Additionally, it is preferred that the composition is 85%, more preferably 95%, and most preferably 99% free of any protein that does not have lysosomal enzyme activity. To the extent that albumin is present in the composition, exemplary compositions of the invention are substantially free of conjugates comprising a poly(ethylene glycol) polymer linking a residue of a lysosomal enzyme moiety to albumin.

Control of the desired number of polymers for any given moiety can be achieved by selecting the proper polymeric reagent, the ratio of polymeric reagent to the lysosomal enzyme moiety, temperature, pH conditions, and other aspects of the conjugation reaction. In addition, reduction or elimination of the undesired conjugates (e.g., those conjugates having four or more attached polymers) can be achieved through purification means.

For example, the polymer-lysosomal enzyme moiety conjugates can be purified to obtain/isolate different conjugated species. Specifically, the product mixture can be purified to obtain an average of anywhere from one, two, three, four, five or more PEGs per lysosomal enzyme moiety, typically one, two or three PEGs per lysosomal enzyme moiety. The strategy for purification of the final conjugate reaction mixture will depend upon a number of factors, including, for example, the molecular weight of the polymeric reagent employed, the particular lysosomal enzyme moiety, the desired dosing regimen, and the residual activity and in vivo properties of the individual conjugate(s).

If desired, conjugates having different molecular weights can be isolated using gel filtration chromatography and/or ion exchange chromatography. That is to say, chromatography is used to fractionate differently numbered polymer-to-lysosomal enzyme moiety ratios (e.g., 1-mer, 2-mer, 3-mer, and so forth, wherein “1-mer” indicates 1 polymer to lysosomal enzyme moiety, “2-mer” indicates two polymers to lysosomal enzyme moiety, and so on) on the basis of their differing molecular weights (where the difference corresponds essentially to the average molecular weight of the water-soluble polymer portion). For example, in an exemplary reaction where a 35,000 Dalton protein is randomly conjugated to a polymeric reagent having a molecular weight of about 20,000 Daltons, the resulting reaction mixture may contain unmodified enzyme (having a molecular weight of about 35,000 Daltons), monoPEGylated protein (having a molecular weight of about 55,000 Daltons), diPEGylated protein (having a molecular weight of about 75,000 Daltons), and so forth.

While this approach can be used to separate PEG and other polymer-lysosomal enzyme moiety conjugates having different molecular weights, this approach is generally ineffective for separating positional isoforms having different polymer attachment sites within the glucocerebrosidase moiety. For example, chromatography can be used to separate from each other mixtures of PEG 1-mers, 2-mers, 3-mers, and so forth, although each of the recovered conjugate compositions may contain PEG(s) attached to different reactive groups (e.g., lysine residues) within the lysosomal enzyme moiety.

Resins suitable for carrying out this type of separation are available from GE Biosciences (Ipsala Sweden). Selection of a particular column will depend upon the desired fractionation range desired. Elution is generally carried out using a suitable buffer, such as phosphate, acetate, or the like. The collected fractions may be analyzed by a number of different methods, for example, (i) absorbance at 280 nm for protein content, (ii) dye-based protein analysis using bovine serum albumin (BSA) as a standard, (iii) iodine testing for PEG content (Sims et al. (1980) Anal. Biochem, 107:60-63), (iv) sodium dodecyl sulfate polyacrylamide gel electrophoresis (SDS PAGE), followed by staining with barium iodide, and (v) high performance liquid chromatography (HPLC).

Separation of positional isoforms is carried out by reverse phase chromatography using a reverse phase-high performance liquid chromatography (RP-HPLC) using a suitable column (e.g., a C18 column or C3 column, available commercially from companies such as Amersham Biosciences or Vydac) or by ion exchange chromatography using an ion exchange column, e.g., a Sepharose™ ion exchange column available from Amersham Biosciences. Either approach can be used to separate polymer-active agent isomers having the same molecular weight (i.e., positional isoforms).

The compositions are preferably substantially free of proteins that do not have lysosomal enzyme activity. In addition, the compositions preferably are substantially free of all other noncovalently attached water-soluble polymers. In some circumstances, however, the composition can contain a mixture of polymer-lysosomal enzyme moiety conjugates and unconjugated lysosomal enzyme moiety.

Optionally, the composition of the invention further comprises a pharmaceutically acceptable excipient. If desired, the pharmaceutically acceptable excipient can be added to a conjugate to form a composition.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The composition can also include an antimicrobial agent for preventing or deterring microbial growth. Nonlimiting examples of antimicrobial agents suitable for one or more embodiments of the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the conjugate or other components of the preparation. Suitable antioxidants for use in one or more embodiments of the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the conjugate (i.e., the conjugate formed between the lysosomal enzyme moiety and the polymeric reagent) in the composition will vary depending on a number of actors, but will optimally be a therapeutically effective dose when the composition is stored in a unit dose container (e.g., a vial). In addition, the pharmaceutical preparation can be housed in a syringe. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A.H., Handbook of Pharmaceutical Excipients, 3^{rd} Edition, American Pharmaceutical Association, Washington, D.C., 2000.

The compositions encompass all types of formulations and in particular those that are suited for infusion, injection, e.g., powders or lyophilates that can be reconstituted as well as liquids. Examples of suitable diluents for reconstituting solid compositions prior to injection include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

The compositions of one or more embodiments of the present invention are typically, although not necessarily, administered via injection and are therefore generally liquid solutions or suspensions immediately prior to administration. The pharmaceutical preparation can also take other forms such as syrups, creams, ointments, tablets, powders, and the like. Other modes of administration are also included, such as pulmonary, rectal, transdermal, transmucosal, oral, intrathecal, subcutaneous, intra-arterial, and so forth.

The invention also provides a method for administering a conjugate as provided herein to a patient suffering from aLSD. The method comprises administering to a patient, generally via infusion, a therapeutically effective amount of the conjugate (preferably provided as part of a pharmaceutical composition). As previously described, the conjugates can be administered by any one of a number of routes of administration, depending upon its formulation. Advantageously, the conjugate can be administered by intramuscular or by subcutaneous injection, wherein the current means of administrating a lysosomal enzyme by enzyme replacement therapy requires intravenous infusion. Thus, the present disclosure provides methods for administering an ERT composition (e.g., a composition comprising a conjugate as described herein) to a patient suffering from a lysosomal storage disease where the administration is performed (a) outside a current or previously licensed medical facility and (b) by the patient. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

Uses

The conjugates provided herein may be used to treat any lysosomal storage disease or related condition that can be remedied or prevented or whose clinical manifestations can be lessened in severity or their progression slowed by administration of the lysosomal enzyme per se. Administration is typically to a mammalian, i.e., human or non-human, subject. Those of ordinary skill in the art will appreciate which conditions a specific conjugate can effectively treat. For example, a glucocerebrosidase conjugate can be used either alone or in combination with other pharmacotherapy to treat patients suffering Gaucher's disease. The conjugates described herein, e.g., of various lysosomal enzyme moieties, and the conditions which such polymer conjugates are useful in treating are described generally in Table 1, although such table in note meant to be exhaustive. For example, administration of a glucocerebrosidase conjugate will be used to treat patients with Type I Gaucher's disease, where clinical manifestations of the disease may include any one of more of the following: anemia, thrombocytopenia, bone disease, hepatomegaly, and splenomegaly. Advantageously, the conjugate can be administered to the patient prior to, simultaneously with, or after administration of another active agent. Similarly, a conjugate of a lysosomal enzyme as provided in Table 1 will be administered to treat the corresponding lysosomal storage disease condition as described in Table 1.

Preferably, a conjugate as provided herein is used to treat a lysosomal storage disorder selected from Gaucher disease (glucocerebrosidase conjugate), Hurler and Hurler-Scheie forms of MPS I (α-iduronidase conjugate), Fabry disease (α-galactosidase conjugate), MPS VI (N-acetylgalactosamine 4-sulfatase conjugate) Pompe disease (α-glucosidase conjugate), and Hunter syndrome (MPS II, iduronate-2-sulfatase conjugate).

Gaucher's disease is the most common of the lysosomal storage diseases, which as a whole, are rare. Gaucher's disease is caused by a deficiency of glucocerebrosidase. Gaucher's disease shows autosomal recessive inheritance, and affects both males and females. There are three types of Gaucher's disease classified as types 1, 2 and 3. Type 1 is the most common; patients suffering from type 1 Gaucher's disease usually bruise easily and experience fatigue due to anemia and low blood platelets. Then also have an enlarged liver and spleen, skeletal disorders, and in some instances, lung and kidney impairment. There are no signs of brain involvement, and symptoms can occur at any age. In type 2 Gaucher's disease, liver and spleen enlargement are apparent by 3 months of age. Patients have extensive and progressive brain damage and typically die by two years of age. In type 3, liver and spleen enlargement is variable, and signs of brain involvement (e.g. seizures) gradually become apparent. Types 2 and 3 account for only about 5 percent of Gaucher's disease. Types 1 and 3 are typically treatable by enzyme replacement therapy, e.g., by administering a glucocerebrosidase polymer conjugate.

Mucopolysaccharidosis type I (Hurler syndrome) is a rare genetic disorder caused by a deficiency of alpha-L-iduronidase, which breaks down glycoaminoglycans. Symptoms can range from mild to severe, depending upon the subtype. Other subtypes include MPS I H-S (Hurler-Scheie syndrome) and MPS I A (Scheie syndrome). Symptoms of Hurler syndrome most often appear between the ages of 3 and 8. Infants with severe Hurler syndrome appear normal at birth, although facial symptoms may become more noticeable during the first two years of life. Symptoms include thick, coarse facial features with a low nasal bridge, halted growth, progressive mental retardation, cloudy corneas, deafness, joint disease, heart valve problems, and abnormal spinal skeletal features. Children born with a mild form of the disease, known as MPS I A, have normal intelligence and may live to adulthood. In yet another form known as MPS I H-S, subjects suffering from this form have normal intelligence and mild to severe physical symptoms. Administration of a water-soluble polymer conjugate of alpha-L-iduronidase is useful to treat (i.e., relieve one or more symptoms) caused by MPS I.

Fabry disease (also known as Anderson-Fabry disease) is an inherited lysosomal storage disorder caused by a deficiency of alpha-galactosidase A (also referred to as ceramidetrihexosidase). As a result, the glycolipid, globotriaosylceramide (GB-3 or GL-3), accumulates in the blood, blood vessels, and organs of the body, leading to impairment of proper function. Accumulation of GL-3 in the blood vessels causes the vessels to become narrower, reducing flow to tissues in the body. Symptoms of Fabry disease usually begin during childhood or adolescence and include pain and burning sensations in the hands and feet, angiokeratomas (skin lesions), corneal cloudiness, kidney and heart complications, abdominal discomfort, and back pain. Enzyme replacement therapy, i.e., administration of agalsidase alpha (alpha galactosidase) or agalsidase beta, is effective to treat Fabry disease. Administration of a water soluble polymer conjugate of an alpha-galactosidase A moiety can be used, e.g., for treating Fabry disease. The conjugates provided herein are used to reduce GL-3 deposition in capillary endothelium of the kidney and certain other cell types. Moreover, administration of a polymer conjugate as described herein can be effective to reduce or eliminate serious and common adverse infusion reactions to alpha-galactosidase A (or any other lysosomal storage enzyme as provided herein) such as chills, pyrexia, feeling hot or cold, dyspnea, nausea, flushing, headache, vomiting, paresthesia, fatigue, pruritus, pain in extremities, hypertension, chest pain, throat tightness, abdominal pain, dizziness, tachycardia, nasal congestion, diarrhea, edema peripheral, myalgia, back pain, pallor, bradycardia, urticaria, hypotension, face edema, and rash.

Maroteaux-Lamy syndrome (MPS VI) is caused by a deficiency of N-acetylgalactosamine 4-sulfatase (arylsulfatase B), an enzyme normally required for the breakdown of glycosaminoglycans. MPS VI is inherited in an autosomal recessive manner, affecting males and females equally. In most cases, both parents of an affected child are asymptomatic carriers of the disease. MPS VI is a clinically heterogeneous disease with a wide variation in the rate of disease progression, the severity of symptoms, and the organ systems affected. MPS VI does not typically affect intelligence level. While patients with a rapidly progressing clinical presentation of MPS VI are usually diagnosed by one to five years of age, those with the more slowly progressing disease may be misdiagnosed. Over time the disease progresses, and depending on the degree of enzyme deficiency, patients experience severe disabilities and possibly early death. Symptoms associated with MPS VI include short stature, large head, progressively coarse facial features, communicating hydrocephalus, spinal cord compression, enlargement of the liver and spleen, sleep apnea, carpal tunnel syndrome and corneal clouding. As MPS VI progresses, patients experience increasingly impaired endurance, eventually leading to severe disability. ERT has been approved for the treatment of MPS VI. Administration of a water-soluble polymer conjugate of acetylgalactosamine 4-sulfatase is useful to treat (i.e., relieve one or more symptoms caused by) MPS VI.

Pompe disease (also called Glycogen storage disease type II or acid maltase deficiency) is caused by a deficiency in the enzyme acid maltase (acid alpha-glucosidase or GAA). Acid maltase is needed to break down glycogen. Pompe disease is the only glycogen storage disease with a defect in lysosomal metabolism, and was the first glycogen storage disease to be identified, in 1932. Pompe disease is estimated to occur in about 1 in 40,000-300,000 births. It has an autosomal recessive inheritance pattern and is an often fatal disorder that disables the heart and muscles. Early onset (infantile) Pompe disease is the result of complete or near complete deficiency of GAA. Symptoms begin in the first months of life, with feeding problems, poor weight gain, muscle weakness, floppiness, and head lag. Respiratory difficulties are often complicated by lung infections. The heart is grossly enlarged. More than half of all infants with Pompe disease also have enlarged tongues. Most babies with Pompe disease die from cardiac or respiratory complications before their first birthday. Late onset (juvenile/adult) Pompe disease is the result of a partial deficiency of GAA. Onset can be as early as the first decase of childhood or as late as the sixth decade of adulthood. The primary symptom is muscle weakness progressing to respiratory weakness and death from respiratory failure after a course lasting several years. Administration of a water-soluble polymer conjugate of acid alpha-glucosidase is useful to treat Pompe disease. The conjugates provided herein may decrease heart size, maintain normal heart function, improve muscle function, tone, and strength, and reduce glycogen accumulation.

Hunter syndrome (MPS II) is caused by the deficiency or absence of the enzyme iduronate-2-sulfatase (IDS). IDS is required for the lysosomal degradation of the glycosamino glycans heparin sulfate and dermatin sulfate. The gene encoding IDS is located on the X-chromosome. Accordingly, Hunter syndrome is an X-linked recessive disorder that primarily affects males. In people with Hunter syndrome, the IDS enzyme is either partially or completely inactive. There are two subtypes of Hunter syndrome, MPS IIA and MPS IIB. Type MPS IIAa is early onset Hunter syndrome and is the more severe of the two types. It usually appears around age 2 and up to age 4. This form of the disorder may result in profound mental retardation by late childhood. Children with this form usually don't survive beyond their teens. Symptoms of MPS IIA include, in part, coarse facial features including thickening of the lips, tongue and nostrils, abnormal bone size or shape, enlarged internal organs such as the liver and spleen, resulting in a distended abdomen, respiratory difficulties, cardiovascular disorders, such as progressive thickening of heart valves, hypertension and obstruction of blood vessels, and vision loss or impairment. MPS IIB (late-onset) is milder and causes less severe symptoms. It is usually diagnosed after age 10, but may not be detected until adulthood. Intellectual and social development usually is nearly normal, but the condition may affect verbal and reading skills. Symptoms include abnormal bone size or shape, somewhat stunted growth, poor peripheral vision, joint stiffness, hearing loss and sleep apnea. Administration of the water-soluble conjugates of the IDS enzyme, as provided herein, is useful to treat of Hunter syndrome.

The actual dose to be administered will vary depending upon the age, weight, and general condition of the subject as well as the severity of the condition being treated, the judgment of the health care professional, and conjugate being administered. Therapeutically effective amounts can be determined by those skilled in the art, e.g., by standard clinical techniques. Generally, a therapeutically effective amount will range from about 0.001 mg to 100 mg, preferably in doses from 0.01 mg/day to 75 mg/day, and more preferably in doses from 0.10 mg/day to 50 mg/day.

The dosage of any given conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration once daily, three times weekly, twice weekly, once weekly, twice monthly, once monthly, and any combination thereof. Once the clinical endpoint has been achieved, if achieved, dosing of the composition may be halted. The administration for a single individual need not be a fixed interval, but may change over time, depending upon the needs of the individual.

One advantage of administering certain conjugates described herein is that individual water-soluble polymer portions can be cleaved when a hydrolytically degradeable linkage is incorporated between the residue of a lysosomal enzyme moiety and the water-soluble polymer. Such a result is advantageous when clearance from the body is potentially a problem because of the polymer size. Optimally, cleavage of each water-soluble polymer portion is facilitated through the use of physiologically cleavable and/or enzymatically degradable linkages such as amide, carbonate or ester-containing linkages. In this way, clearance of the conjugate (via cleavage of individual water-soluble polymer portions) can be modulated by selecting the polymer molecular size and the type functional group that would provide the desired clearance properties. One of ordinary skill in the art can determine the proper molecular size of the polymer as well as the cleavable functional group. For example, one of ordinary skill in the art, using routine experimentation, can determine a proper molecular size and cleavable functional group by first preparing a variety of polymer derivatives with different polymer weights and cleavable functional groups, and then obtaining the clearance profile (e.g., through periodic blood or urine sampling) by administering the polymer derivative to a patient and taking periodic blood and/or urine sampling. Once a series of clearance profiles have been obtained for each tested conjugate, a suitable conjugate can be identified.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All articles, books, patents and other publications referenced herein are hereby incorporated by reference in their entireties.

### EXPERIMENTAL

The practice of the invention will employ, unless otherwise indicated, conventional techniques of organic synthesis, biochemistry, protein purification and the like, which are within the skill of the art. Such techniques are fully explained in the literature. See, for example, J. March, Advanced Organic Chemistry: Reactions Mechanisms and Structure, 4th Ed. (New York: Wiley-Interscience, 1992), supra.

In the following prophetic examples, efforts have been made to ensure accuracy with respect to numbers used (e.g., amounts, temperatures, etc.) but some experimental error and deviation should be taken into account. Unless indicated otherwise, temperature is in degrees C. and pressure is at or near atmospheric pressure at sea level. Each of the following examples is considered to be instructive to one of ordinary skill in the art for carrying out one or more of the embodiments described herein.

An aqueous solution (“stock rGC solution”) comprising the glucocerebrosidase moiety corresponding to the amino acid sequence of SEQ ID NO 1 (rGC) is obtained for use in the examples.

SDS-PAGE Analysis

Samples can be analyzed by sodium dodecyl sulfate-polyacrylamide gel electrophoresis (SDS-PAGE) using Bio-Rad system (Mini-PROTEAN III Precast Gel Electrophoresis System). Samples are mixed with sample buffer. Then, the prepared samples can be loaded onto a gel and run for approximately thirty minutes.

Anion Exchange Chromatography

A Hitrap Q Sepharose FF anion exchange column (5 ml, Amersham Biosciences) can be used with the AKTAprime system (Amersham Biosciences) to purify the prepared PEG-rGC conjugates. For each conjugate solution prepared, the conjugate solution is loaded on a column that is pre-equilibrated in 20 mM Tris buffer, pH 7.5 (buffer A) and is then washed with nine column volumes of buffer A to remove any unreacted PEG reagent. Subsequently, a gradient of buffer A with 0-100% buffer B (20 mM Tris with 0.5 M NaCl buffer, pH 7.5) can be used. The eluent is monitored by UV detector at 280 nm. Any higher-mers (e.g., 11-mers, 10-mers, and so forth) will elute first, followed by increasingly smaller and smaller conjugates (e.g, 5-mers and 4-mers, and so forth), until 1-mers, and finally, unconjugated rGC species elute. The fractions can be pooled and the purity of the individual conjugate can be determined by SEC-HPLC.

SEC-HPLC Analysis

Size exclusion chromatography (SEC-HPLC) analysis can be performed on an Agilent 1100 HPLC system (Agilent). Samples are analyzed using a Shodex protein KW-804 column (300×8 mm, Phenomenex), and a mobile phase consisting of 90% phosphate buffered saline and 10% ethanol, pH 7.4. The flow rate for the column can be 0.5 ml/min. Eluted protein and PEG-protein conjugates can be detected using UV at 280 nm.

### Example 1

### PEGylation of rGC with Branched mPEG-N-Hydroxysuccinimide Derivative, 40 kDa

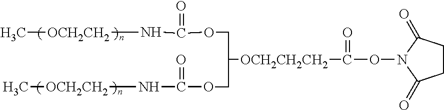

### Branched mPEG-N-Hydroxysuccinimide Derivative, 40 kDa, (“mPEG2-NHS”)

mPEG2-NHS, 40 kDa, stored at −20° C. under argon, is warmed to ambient temperature. A five-fold excess (relative to the amount of rGC in a measured aliquot of the stock rGC solution) of the warmed mPEG2-NHS is dissolved in 2 mM HCl to form a 10% reagent solution. The 10% reagent solution is quickly added to the aliquot of stock rGC solution and is mixed well. After the addition of the mPEG2-NHS, the pH of the reaction mixture is determined and adjusted to 7.0 to 8.0 using conventional techniques. To allow for coupling of the mPEG2-NHS to rGC via an amide linkage, the reaction solution is stirred for five hours at room temperature in the dark, thereby resulting in a conjugate solution. The reaction is quenched with glycine.

mPEG2-NHS is found to provide a relatively large molecular volume of active N-hydroxysuccinimide (“NHS”) ester, which selectively reacts with lysine and terminal amines.

Using this same approach, other conjugates are prepared (i) using mPEG2-NHS having other weight average molecular weights, and (ii) using other lysosomal enzymes as described herein.

### Example 2

### PEGylation of rGC with Linear mPEG-Succinimidyl α-Methylbutanoate Derivative, 30 kDa

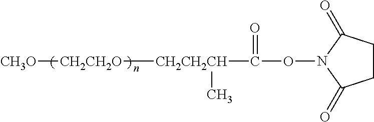

### Linear mPEG-Succinimidyl α-Methylbutanoate Derivative, 30 kDa (“mPEG-SMB”)

mPEG-SMB, 30 kDa, stored at −20° C. under argon, is warmed to ambient temperature. A five-fold excess (relative to the amount of rGC in a measured aliquot of the stock rGC solution) of the warmed mPEG-SMB is dissolved in 2 mM HCl to form a 10% reagent solution. The 10% reagent solution is quickly added to the aliquot of stock rGC solution and is mixed well. After the addition of the mPEG-SMB, the pH of the reaction mixture is determined and adjusted to 7.0 to 8.0 using conventional techniques. To allow for coupling of the mPEG-SMB to rGC via an amide linkage, the reaction solution is stirred for five hours at room temperature in the dark, thereby resulting in a conjugate solution. The reaction is quenched with glycine.

The mPEG-SMB derivative is found to provide a sterically hindered active NHS ester, which selectively reacts with lysine and terminal amines.

Using this same approach, other conjugates are prepared (i) using mPEG-SMB having other weight average molecular weights, and (ii) using other lysosomal enzymes as described herein.

### Example 3

### PEGylation of rGC with Linear mPEG-Butyraldehyde Derivative, 30 kDa

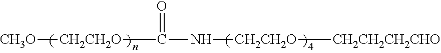

### Linear mPEG-Butyraldehyde Derivative, 30 kDa (“mPEG-ButyrALD”)

mPEG-ButyrALD, 30 kDa, stored at −20° C. under argon, is warmed to ambient temperature. An eight-fold excess (relative to the amount of rGC in a measured aliquot of the stock rGC) of the warmed mPEG-ButryALD is dissolved in 10 mM sodium phosphate (pH 7.2) to form a 10% reagent solution. The 10% reagent solution is quickly added to the aliquot of stock rGC solution and is mixed well. After the addition of the mPEG-ButryALD, the pH of the reaction mixture is determined and is adjusted to around 5.5 using conventional techniques, followed by mixing for thirty minutes. A reducing agent, sodium cyanoborohydride (NaCNBH₃), is then added at sixty to seventy molar excess relative to the rGC (with the pH tested and adjusted using conventional techniques to ensure a pH of around 5.5). The reaction solution is thereafter stirred for about ten minutes and placed overnight in a 3-8° C. cold room to ensure coupling via a secondary amine linkage to thereby form a conjugate solution. Using this reagent at a higher pH (e.g., 7.2 versus around 5.5) is believed to yield a secondary amine linkage, but with potentially fewer conjugation events at the N-terminus. The reaction is quenched with glycine.

The aldehyde group of mPEG-ButyrALD is found to react with the primary amines associated with rGC and covalently bond to them via secondary amine upon reduction by a reducing reagent such as sodium cyanoborohydride.

Using this same approach, other conjugates are prepared (i) using mPEG-BuryrALD having other weight average molecular weights, and (ii) using other lysosomal enzymes as described herein.

### Example 4

### PEGylation of rGC with Branched mPEG-Butyraldehyde Derivative, 40 kDa

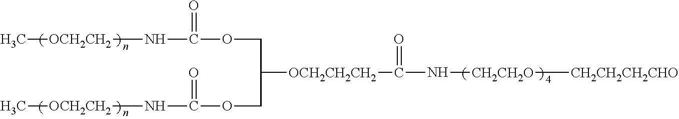

### Branched mPEG-Butyraldehyde Derivative, 40 kDa (“mPEG2-ButyrALD”)

mPEG-ButyrALD, 40 kDa, stored at −20° C. under argon, is warmed to ambient temperature. A ten-fold excess (relative to the amount of r glucocerebrosidase in a measured aliquot of the stock rGC solution) of the warmed mPEG-ButryALD is dissolved in 10 mM sodium phosphate (pH 7.2) to form a 10% reagent solution. The 10% reagent solution is quickly added to the stock rGC solution and is mixed well. After the addition of the mPEG2-ButryALD, the pH of the reaction mixture is determined and is adjusted to around 5.5 using conventional techniques, followed by mixing for thirty minutes. A reducing agent, sodium cyanoborohydride (NaCNBH₃), is added at about seventy molar excess relative the rGC (with the pH tested and adjusted using conventional techniques to ensure a pH of about around 5.5). The reaction solution is thereafter stirred for about ten minutes and placed overnight in a 3-8° C. cold room to ensure coupling via a secondary amine linkage to thereby form a conjugate solution. The reaction is quenched with glycine.

The aldehyde group of mPEG2-ButyrALD is found to react with the primary amines associated with rGC and covalently bond to them via secondary amine upon reduction by a reducing reagent such as sodium cyanoborohydride.

Using this same approach, other conjugates are prepared (i) using mPEG2-BuryrALD having other weight average molecular weights, and (ii) using other lysosomal enzymes as described herein.

### Example 5

### PEGylation of rGC with mPEG SBC (to form a Conjugate with a Cleavable Bond)

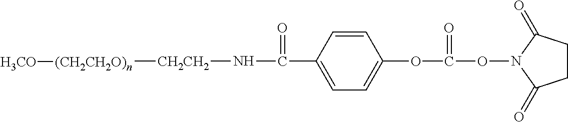

### mPEG SBC

The PEG reagent, mPEG SBC having a weight average molecular weight of 5,000 Daltons, is warmed from −20° C. to room temperature in a dessicator. A five-fold excess (relative to the amount of rGC in a measured aliquot of the stock rGC solution) of the warmed mPEG SBC is dissolved in 2 mM HCl to form an mPEG SBC solution. The mPEG SBC solution is added to the aliquot of stock rGC solution and is mixed well. After the addition of the mPEG CSB, the pH of the reaction mixture is determined and adjusted to around 7.0 using conventional techniques. To allow for coupling, the reaction is stirred for five hours at room temperature, thereby resulting in a conjugate solution. The reaction is quenched with glycine.

The non-peptidic, water-soluble polymer is attached at amine groups. The conjugate contains a cleavable linkage.

Using this same approach, other conjugates are prepared (i) using mPEG SBC having other weight average molecular weights, and (ii) using other lysosomal enzymes as described herein.

### Example 6

### PEGylation of rGC with mPEG-MAL, 20 kDa

mPEG-Maleimide having a molecular weight of 20,000 Daltons is obtained from Nektar Therapeutics, (Huntsville, Ala.). The basic structure of the polymeric reagent is provided below:

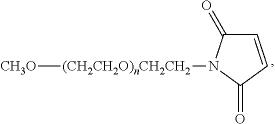

### mPEG-MAL, 20 kDa

rGC is dissolved in buffer. To this protein solution is added a 3-5 fold molar excess of mPEG-MAL, 20 kDa. The mixture is stirred at room temperature under an inert atmosphere for several hours. Analysis of the reaction mixture reveals successful conjugation of rGC.

Using this same approach, other conjugates are prepared (i) using mPEG-MAL having other weight average molecular weights, and (ii) using other lysosomal enzymes as described herein.

### Example 7

### In-Vitro Activity of Exemplary (rGC)-PEG Conjugates

The in-vitro activities of the (rGC)-PEG and other conjugates described in the preceding Examples are determined. All of the rGC and other lysosomal enzyme moiety conjugates are believed to be bioactive.

### SEQUENCE LISTING

```
SEQ ID NO: 1
Met_{(n′′′)}
Ala Arg Pro Cys Ile Pro Lys Ser Phe Gly Tyr Ser Ser Val Val Cys
1 5 10 15
Val Cys Asn Ala Thr Tyr Cys Asp Ser Phe Asp Pro Pro Thr Phe Pro
                        20 25 30
Ala Leu Gly Thr Phe Ser Arg Tyr Glu Ser Thr Arg Ser Gly Arg Arg
                35 40 45
Met Glu Leu Ser Met Gly Pro Ile Gln Ala Asn His Thr Gly Thr Gly
        50 55 60
Leu Leu Leu Thr Leu Gln Pro Glu Gln Lys Phe Gln Lys Val Lys Gly
65 70 75 80
Phe Gly Gly Ala Met Thr Asp Ala Ala Ala Leu Asn Ile Leu Ala Leu
                                85 90 95
Ser Pro Pro Ala Gln Asn Leu Leu Leu Lys Ser Tyr Phe Ser Glu Glu
                        100 105 110
Gly Ile Gly Tyr Asn Ile Ile Arg Val Pro Met Ala Ser Cys Asp Phe
                115 120 125
Ser Ile Arg Thr Tyr Thr Tyr Ala Asp Thr Pro Asp Asp Phe Gln Leu
        130 135 140
His Asn Phe Ser Leu Pro Glu Glu Asp Thr Lys Leu Lys Ile Pro Leu
145 150 155 160
Ile His Arg Ala Leu Gln Leu Ala Gln Arg Pro Val Ser Leu Leu Ala
                                165 170 175
Ser Pro Trp Thr Ser Pro Thr Trp Leu Lys Thr Asn Gly Ala Val Asn
                        180 185 190
Gly Lys Gly Ser Leu Lys Gly Gln Pro Gly Asp Ile Tyr His Gln Thr
                195 200 205
Trp Ala Arg Tyr Phe Val Lys Phe Leu Asp Ala Tyr Ala Glu His Lys
        210 215 220
Leu Gln Phe Trp Ala Val Thr Ala Glu Asn Glu Pro Ser Ala Gly Leu
225 230 235 240
Leu Ser Gly Tyr Pro Phe Gln Cys Leu Gly Phe Thr Pro Glu His Gln
                                245 250 255
Arg Asp Phe Ile Ala Arg Asp Leu Gly Pro Thr Leu Ala Asn Ser Thr
                        260 265 270
His His Asn Val Arg Leu Leu Met Leu Asp Asp Gln Arg Leu Leu Leu
                275 280 285
Pro His Trp Ala Lys Val Val Leu Thr Asp Pro Glu Ala Ala Lys Tyr
        290 295 300
Val His Gly Ile Ala Val His Trp Tyr Leu Asp Phe Leu Ala Pro Ala
305 310 315 320
Lys Ala Thr Leu Gly Glu Thr His Arg Leu Phe Pro Asn Thr Met Leu
                                325 330 335
Phe Ala Ser Glu Ala Cys Val Gly Ser Lys Phe Trp Glu Gln Ser Val
                        340 345 350
Arg Leu Gly Ser Trp Asp Arg Gly Met Gln Tyr Ser His Ser Ile Ile
                355 360 365
Thr Asn Leu Leu Tyr His Val Val Gly Trp Thr Asp Trp Asn Leu Ala
        370 375 380
Leu Asn Pro Glu Gly Gly Pro Asn Trp Val Arg Asn Phe Val Asp Ser
385 390 395 400
Pro Ile Ile Val Asp Ile Thr Lys Asp Thr Phe Tyr Lys Gln Pro Met
                                405 410 415
Phe Tyr His Leu Gly His Phe Ser Lys Phe Ile Pro Glu Gly Ser Gln
                        420 425 430
Arg Val Gly Leu Val Ala Ser Gln Lys Asn Asp Leu Asp Ala Val Ala
                435 440 445
Leu Met His Pro Asp Gly Ser Ala Val Val Val Val Leu Asn Arg Ser
        450 455 460
Ser Lys Asp Val Pro Leu Thr Ile Lys Asp Pro Ala Val Gly Phe Leu
465 470 475 480
Glu Thr Ile Ser Pro Gly Tyr Ser Ile His Thr Tyr Leu Trp His Arg
                                485 490 495
Gln Arg
wherein _{n′′′} = 0 or 1
```

```
SEQ ID NO: 2
Met_{(n′′′)}
Ala Arg Pro Cys Ile Pro Lys Ser Phe Gly Tyr Ser Ser Val Val Cys
1 5 10 15
Val Cys Asn Ala Thr Tyr Cys Asp Ser Phe Asp Pro Pro Thr Phe Pro
                        20 25 30
Ala Leu Gly Thr Phe Ser Arg Tyr Glu Ser Thr Arg Ser Gly Arg Arg
                35 40 45
Met Glu Leu Ser Met Gly Pro Ile Gln Ala Asn His Thr Gly Thr Gly
        50 55 60
Leu Leu Leu Thr Leu Gln Pro Glu Gln Lys Phe Gln Lys Val Lys Gly
65 70 75 80
Phe Gly Gly Ala Met Thr Asp Ala Ala Ala Leu Asn Ile Leu Ala Leu
                                85 90 95
Ser Pro Pro Ala Gln Asn Leu Leu Leu Lys Ser Tyr Phe Ser Glu Glu
                        100 105 110
Gly Ile Gly Tyr Asn Ile Ile Arg Val Pro Met Ala Ser Cys Asp Phe
                115 120 125
Ser Ile Arg Thr Tyr Thr Tyr Ala Asp Thr Pro Asp Asp Phe Gln Leu
        130 135 140
His Asn Phe Ser Leu Pro Glu Glu Asp Thr Lys Leu Lys Ile Pro Leu
145 150 155 160
Ile His Arg Ala Leu Gln Leu Ala Gln Arg Pro Val Ser Leu Leu Ala
                                165 170 175
Ser Pro Trp Thr Ser Pro Thr Trp Leu Lys Thr Asn Gly Ala Val Asn
                        180 185 190
Gly Lys Gly Ser Leu Lys Gly Gln Pro Gly Asp Ile Tyr His Gln Thr
                195 200 205
Trp Ala Arg Tyr Phe Val Lys Phe Leu Asp Ala Tyr Ala Glu His Lys
        210 215 220
Leu Gln Phe Trp Ala Val Thr Ala Glu Asn Glu Pro Ser Ala Gly Leu
225 230 235 240
Leu Ser Gly Tyr Pro Phe Gln Cys Leu Gly Phe Thr Pro Glu His Gln
                                245 250 255
Arg Asp Phe Ile Ala Arg Asp Leu Gly Pro Thr Leu Ala Asn Ser Thr
                        260 265 270
His His Asn Val Arg Leu Leu Met Leu Asp Asp Gln Arg Leu Leu Leu
                275 280 285
Pro His Trp Ala Lys Val Val Leu Thr Asp Pro Glu Ala Ala Lys Tyr
        290 295 300
Val His Gly Ile Ala Val His Trp Tyr Leu Asp Phe Leu Ala Pro Ala
305 310 315 320
Lys Ala Thr Leu Gly Glu Thr His Arg Leu Phe Pro Asn Thr Met Leu
                                325 330 335
Phe Ala Ser Glu Ala Cys Val Gly Ser Lys Phe Trp Glu Gln Ser Val
                        340 345 350
Arg Leu Gly Ser Trp Asp Arg Gly Met Gln Tyr Ser His Ser Ile Ile
                355 360 365
Thr Asn Leu Leu Tyr His Val Val Gly Trp Thr Asp Trp Asn Leu Ala
        370 375 380
Leu Asn Pro Glu Gly Gly Pro Asn Trp Val Arg Asn Phe Val Asp Ser
385 390 395 400
Pro Ile Ile Val Asp Ile Thr Lys Asp Thr Phe Tyr Lys Gln Pro Met
                                405 410 415
Phe Tyr His Leu Gly His Phe Ser Lys Phe Ile Pro Glu Gly Ser Gln
                        420 425 430
Arg Val Gly Leu Val Ala Ser Gln Lys Asn Asp Leu Asp Ala Val Ala
                435 440 445
Leu Met His Pro Asp Gly Ser Ala Val Val Val Val Leu Asn Arg Ser
        450 455 460
Ser Lys Asp Val Pro Leu Thr Ile Lys Asp Pro Ala Val Gly Phe Leu
465 470 475 480
Glu Thr Ile Ser Pro Gly Tyr Ser Ile His Thr Tyr Leu Trp His Arg
                                485 490 495
Gln
wherein _{n′′′} = 0 or 1
```

```
SEQ ID NO: 3
Met_{(n′′′)}
Ala Arg Pro Cys Ile Pro Lys Ser Phe Gly Tyr Ser Ser Val Val Cys
1 5 10 15
Val Cys Asn Ala Thr Tyr Cys Asp Ser Phe Asp Pro Pro Thr Phe Pro
                        20 25 30
Ala Leu Gly Thr Phe Ser Arg Tyr Glu Ser Thr Arg Ser Gly Arg Arg
                35 40 45
Met Glu Leu Ser Met Gly Pro Ile Gln Ala Asn His Thr Gly Thr Gly
        50 55 60
Leu Leu Leu Thr Leu Gln Pro Glu Gln Lys Phe Gln Lys Val Lys Gly
65 70 75 80
Phe Gly Gly Ala Met Thr Asp Ala Ala Ala Leu Asn Ile Leu Ala Leu
                                85 90 95
Ser Pro Pro Ala Gln Asn Leu Leu Leu Lys Ser Tyr Phe Ser Glu Glu
                        100 105 110
Gly Ile Gly Tyr Asn Ile Ile Arg Val Pro Met Ala Ser Cys Asp Phe
                115 120 125
Ser Ile Arg Thr Tyr Thr Tyr Ala Asp Thr Pro Asp Asp Phe Gln Leu
        130 135 140
His Asn Phe Ser Leu Pro Glu Glu Asp Thr Lys Leu Lys Ile Pro Leu
145 150 155 160
Ile His Arg Ala Leu Gln Leu Ala Gln Arg Pro Val Ser Leu Leu Ala
                                165 170 175
Ser Pro Trp Thr Ser Pro Thr Trp Leu Lys Thr Asn Gly Ala Val Asn
                        180 185 190
Gly Lys Gly Ser Leu Lys Gly Gln Pro Gly Asp Ile Tyr His Gln Thr
                195 200 205
Trp Ala Arg Tyr Phe Val Lys Phe Leu Asp Ala Tyr Ala Glu His Lys
        210 215 220
Leu Gln Phe Trp Ala Val Thr Ala Glu Asn Glu Pro Ser Ala Gly Leu
225 230 235 240
Leu Ser Gly Tyr Pro Phe Gln Cys Leu Gly Phe Thr Pro Glu His Gln
                                245 250 255
Arg Asp Phe Ile Ala Arg Asp Leu Gly Pro Thr Leu Ala Asn Ser Thr
                        260 265 270
His His Asn Val Arg Leu Leu Met Leu Asp Asp Gln Arg Leu Leu Leu
                275 280 285
Pro His Trp Ala Lys Val Val Leu Thr Asp Pro Glu Ala Ala Lys Tyr
        290 295 300
Val His Gly Ile Ala Val His Trp Tyr Leu Asp Phe Leu Ala Pro Ala
305 310 315 320
Lys Ala Thr Leu Gly Glu Thr His Arg Leu Phe Pro Asn Thr Met Leu
                                325 330 335
Phe Ala Ser Glu Ala Cys Val Gly Ser Lys Phe Trp Glu Gln Ser Val
                        340 345 350
Arg Leu Gly Ser Trp Asp Arg Gly Met Gln Tyr Ser His Ser Ile Ile
                355 360 365
Thr Asn Leu Leu Tyr His Val Val Gly Trp Thr Asp Trp Asn Leu Ala
        370 375 380
Leu Asn Pro Glu Gly Gly Pro Asn Trp Val Arg Asn Phe Val Asp Ser
385 390 395 400
Pro Ile Ile Val Asp Ile Thr Lys Asp Thr Phe Tyr Lys Gln Pro Met
                                405 410 415
Phe Tyr His Leu Gly His Phe Ser Lys Phe Ile Pro Glu Gly Ser Gln
                        420 425 430
Arg Val Gly Leu Val Ala Ser Gln Lys Asn Asp Leu Asp Ala Val Ala
                435 440 445
Leu Met His Pro Asp Gly Ser Ala Val Val Val Val Leu Asn Arg Ser
        450 455 460
Ser Lys Asp Val Pro Leu Thr Ile Lys Asp Pro Ala Val Gly Phe Leu
465 470 475 480
Glu Thr Ile Ser Pro Gly Tyr Ser Ile His Thr Tyr Leu Trp Arg Arg
                                485 490 495
Gln Arg
wherein _{n′′′} = 0 or 1
```

```
SEQ ID NO: 4
Met_{(n′′′)}
Ala Arg Pro Cys Ile Pro Lys Ser Phe Gly Tyr Ser Ser Val Val Cys
1 5 10 15
Val Cys Asn Ala Thr Tyr Cys Asp Ser Phe Asp Pro Pro Thr Phe Pro
                        20 25 30
Ala Leu Gly Thr Phe Ser Arg Tyr Glu Ser Thr Arg Ser Gly Arg Arg
                35 40 45
Met Glu Leu Ser Met Gly Pro Ile Gln Ala Asn His Thr Gly Thr Gly
        50 55 60
Leu Leu Leu Thr Leu Gln Pro Glu Gln Lys Phe Gln Lys Val Lys Gly
65 70 75 80
Phe Gly Gly Ala Met Thr Asp Ala Ala Ala Leu Asn Ile Leu Ala Leu
                                85 90 95
Ser Pro Pro Ala Gln Asn Leu Leu Leu Lys Ser Tyr Phe Ser Glu Glu
                        100 105 110
Gly Ile Gly Tyr Asn Ile Ile Arg Val Pro Met Ala Ser Cys Asp Phe
                115 120 125
Ser Ile Arg Thr Tyr Thr Tyr Ala Asp Thr Pro Asp Asp Phe Gln Leu
        130 135 140
His Asn Phe Ser Leu Pro Glu Glu Asp Thr Lys Leu Lys Ile Pro Leu
145 150 155 160
Ile His Arg Ala Leu Gln Leu Ala Gln Arg Pro Val Ser Leu Leu Ala
                                165 170 175
Ser Pro Trp Thr Ser Pro Thr Trp Leu Lys Thr Asn Gly Ala Val Asn
                        180 185 190
Gly Lys Gly Ser Leu Lys Gly Gln Pro Gly Asp Ile Tyr His Gln Thr
                195 200 205
Trp Ala Arg Tyr Phe Val Lys Phe Leu Asp Ala Tyr Ala Glu His Lys
        210 215 220
Leu Gln Phe Trp Ala Val Thr Ala Glu Asn Glu Pro Ser Ala Gly Leu
225 230 235 240
Leu Ser Gly Tyr Pro Phe Gln Cys Leu Gly Phe Thr Pro Glu His Gln
                                245 250 255
Arg Asp Phe Ile Ala Arg Asp Leu Gly Pro Thr Leu Ala Asn Ser Thr
                        260 265 270
His His Asn Val Arg Leu Leu Met Leu Asp Asp Gln Arg Leu Leu Leu
                275 280 285
Pro His Trp Ala Lys Val Val Leu Thr Asp Pro Glu Ala Ala Lys Tyr
        290 295 300
Val His Gly Ile Ala Val His Trp Tyr Leu Asp Phe Leu Ala Pro Ala
305 310 315 320
Lys Ala Thr Leu Gly Glu Thr His Arg Leu Phe Pro Asn Thr Met Leu
                                325 330 335
Phe Ala Ser Glu Ala Cys Val Gly Ser Lys Phe Trp Glu Gln Ser Val
                        340 345 350
Arg Leu Gly Ser Trp Asp Arg Gly Met Gln Tyr Ser His Ser Ile Ile
                355 360 365
Thr Asn Leu Leu Tyr His Val Val Gly Trp Thr Asp Trp Asn Leu Ala
        370 375 380
Leu Asn Pro Glu Gly Gly Pro Asn Trp Val Arg Asn Phe Val Asp Ser
385 390 395 400
Pro Ile Ile Val Asp Ile Thr Lys Asp Thr Phe Tyr Lys Gln Pro Met
                                405 410 415
Phe Tyr His Leu Gly His Phe Ser Lys Phe Ile Pro Glu Gly Ser Gln
                        420 425 430
Arg Val Gly Leu Val Ala Ser Gln Lys Asn Asp Leu Asp Ala Val Ala
                435 440 445
Leu Met His Pro Asp Gly Ser Ala Val Val Val Val Leu Asn Arg Ser
        450 455 460
Ser Lys Asp Val Pro Leu Thr Ile Lys Asp Pro Ala Val Gly Phe Leu
465 470 475 480
Glu Thr Ile Ser Pro Gly Tyr Ser Ile His Thr Tyr Leu Trp Arg Arg
                                485 490 495
Gln
wherein _{n′′′} = 0 or 1
```

```
SEQ ID NO: 5
Met_{(n′′′)} Glu Phe Ser Ser Pro Ser Arg Glu Glu Cys Pro Lys Pro Leu
Ser
1 5 10 15
Arg Val Ser Ile Met Ala Gly Ser Leu Thr Gly Leu Leu Leu Leu Gln
                        20 25 30
Ala Val Ser Trp Ala Ser Gly Ala Arg Pro Cys Ile Pro Lys Ser Phe
                35 40 45
Gly Tyr Ser Ser Val Val Cys Val Cys Asn Ala Thr Tyr Cys Asp Ser
        50 55 60
Phe Asp Pro Pro Thr Phe Pro Ala Leu Gly Thr Phe Ser Arg Tyr Glu
65 70 75 80
Ser Thr Arg Ser Gly Arg Arg Met Glu Leu Ser Met Gly Pro Ile Gln
                                85 90 95
Ala Asn His Thr Gly Thr Gly Leu Leu Leu Thr Leu Gln Pro Glu Gln
                        100 105 110
Lys Phe Gln Lys Val Lys Gly Phe Gly Gly Ala Met Thr Asp Ala Ala
                115 120 125
Ala Leu Asn Ile Leu Ala Leu Ser Pro Pro Ala Gln Asn Leu Leu Leu
        130 135 140
Lys Ser Tyr Phe Ser Glu Glu Gly Ile Gly Tyr Asn Ile Ile Arg Val
145 150 155 160
Pro Met Ala Ser Cys Asp Phe Ser Ile Arg Thr Tyr Thr Tyr Ala Asp
                                165 170 175
Thr Pro Asp Asp Phe Gln Leu His Asn Phe Ser Leu Pro Glu Glu Asp
                        180 185 190
Thr Lys Leu Lys Ile Pro Leu Ile His Arg Ala Leu Gln Leu Ala Gln
                195 200 205
Arg Pro Val Ser Leu Leu Ala Ser Pro Trp Thr Ser Pro Thr Trp Leu
        210 215 220
Lys Thr Asn Gly Ala Val Asn Gly Lys Gly Ser Leu Lys Gly Gln Pro
225 230 235 240
Gly Asp Ile Tyr His Gln Thr Trp Ala Arg Tyr Phe Val Lys Phe Leu
                                245 250 255
Asp Ala Tyr Ala Glu His Lys Leu Gln Phe Trp Ala Val Thr Ala Glu
                        260 265 270
Asn Glu Pro Ser Ala Gly Leu Leu Ser Gly Tyr Pro Phe Gln Cys Leu
                275 280 285
Gly Phe Thr Pro Glu His Gln Arg Asp Phe Ile Ala Arg Asp Leu Gly
        290 295 300
Pro Thr Leu Ala Asn Ser Thr His His Asn Val Arg Leu Leu Met Leu
305 310 315 320
Asp Asp Gln Arg Leu Leu Leu Pro His Trp Ala Lys Val Val Leu Thr
                                325 330 335
Asp Pro Glu Ala Ala Lys Tyr Val His Gly Ile Ala Val His Trp Tyr
                        340 345 350
Leu Asp Phe Leu Ala Pro Ala Lys Ala Thr Leu Gly Glu Thr His Arg
                355 360 365
Leu Phe Pro Asn Thr Met Leu Phe Ala Ser Glu Ala Cys Val Gly Ser
        370 375 380
Lys Phe Trp Glu Gln Ser Val Arg Leu Gly Ser Trp Asp Arg Gly Met
385 390 395 400
Gln Tyr Ser His Ser Ile Ile Thr Asn Leu Leu Tyr His Val Val Gly
                                405 410 415
Trp Thr Asp Trp Asn Leu Ala Leu Asn Pro Glu Gly Gly Pro Asn Trp
                        420 425 430
Val Arg Asn Phe Val Asp Ser Pro Ile Ile Val Asp Ile Thr Lys Asp
                435 440 445
Thr Phe Tyr Lys Gln Pro Met Phe Tyr His Leu Gly His Phe Ser Lys
        450 455 460
Phe Ile Pro Glu Gly Ser Gln Arg Val Gly Leu Val Ala Ser Gln Lys
465 470 475 480
Asn Asp Leu Asp Ala Val Ala Leu Met His Pro Asp Gly Ser Ala Val
                                485 490 495
Val Val Val Leu Asn Arg Ser Ser Lys Asp Val Pro Leu Thr Ile Lys
                        500 505 510
Asp Pro Ala Val Gly Phe Leu Glu Thr Ile Ser Pro Gly Tyr Ser Ile
                515 520 525
His Thr Tyr Leu Trp Arg¹ Arg Gln Arg_{(n′′′)}
        530 535
wherein each _{n′′′} is indendently 0 or 1, and Arg¹ is either Arg
or His
```

```
SEQ ID NO: 6
Met_{(n′′′)} Ala Gly Ser Leu Thr Gly Leu Leu Leu Leu Gln Ala Val Ser
Trp
1 5 10 15
Ala Ser Gly Ala Arg Pro Cys Ile Pro Lys Ser Phe Gly Tyr Ser Ser
                        20 25 30
Val Val Cys Val Cys Asn Ala Thr Tyr Cys Asp Ser Phe Asp Pro Pro
                35 40 45
Thr Phe Pro Ala Leu Gly Thr Phe Ser Arg Tyr Glu Ser Thr Arg Ser
        50 55 60
Gly Arg Arg Met Glu Leu Ser Met Gly Pro Ile Gln Ala Asn His Thr
65 70 75 80
Gly Thr Gly Leu Leu Leu Thr Leu Gln Pro Glu Gln Lys Phe Gln Lys
                                85 90 95
Val Lys Gly Phe Gly Gly Ala Met Thr Asp Ala Ala Ala Leu Asn Ile
                        100 105 110
Leu Ala Leu Ser Pro Pro Ala Gln Asn Leu Leu Leu Lys Ser Tyr Phe
                115 120 125
Ser Glu Glu Gly Ile Gly Tyr Asn Ile Ile Arg Val Pro Met Ala Ser
        130 135 140
Cys Asp Phe Ser Ile Arg Thr Tyr Thr Tyr Ala Asp Thr Pro Asp Asp
145 150 155 160
Phe Gln Leu His Asn Phe Ser Leu Pro Glu Glu Asp Thr Lys Leu Lys
                                165 170 175
Ile Pro Leu Ile His Arg Ala Leu Gln Leu Ala Gln Arg Pro Val Ser
                        180 185 190
Leu Leu Ala Ser Pro Trp Thr Ser Pro Thr Trp Leu Lys Thr Asn Gly
                195 200 205
Ala Val Asn Gly Lys Gly Ser Leu Lys Gly Gln Pro Gly Asp Ile Tyr
        210 215 220
His Gln Thr Trp Ala Arg Tyr Phe Val Lys Phe Leu Asp Ala Tyr Ala
225 230 235 240
Glu His Lys Leu Gln Phe Trp Ala Val Thr Ala Glu Asn Glu Pro Ser
                                245 250 255
Ala Gly Leu Leu Ser Gly Tyr Pro Phe Gln Cys Leu Gly Phe Thr Pro
                        260 265 270
Glu His Gln Arg Asp Phe Ile Ala Arg Asp Leu Gly Pro Thr Leu Ala
                275 280 285
Asn Ser Thr His His Asn Val Arg Leu Leu Met Leu Asp Asp Gln Arg
        290 295 300
Leu Leu Leu Pro His Trp Ala Lys Val Val Leu Thr Asp Pro Glu Ala
305 310 315 320
Ala Lys Tyr Val His Gly Ile Ala Val His Trp Tyr Leu Asp Phe Leu
                                325 330 335
Ala Pro Ala Lys Ala Thr Leu Gly Glu Thr His Arg Leu Phe Pro Asn
                        340 345 350
Thr Met Leu Phe Ala Ser Glu Ala Cys Val Gly Ser Lys Phe Trp Glu
                355 360 365
Gln Ser Val Arg Leu Gly Ser Trp Asp Arg Gly Met Gln Tyr Ser His
        370 375 380
Ser Ile Ile Thr Asn Leu Leu Tyr His Val Val Gly Trp Thr Asp Trp
385 390 395 400
Asn Leu Ala Leu Asn Pro Glu Gly Gly Pro Asn Trp Val Arg Asn Phe
                                405 410 415
Val Asp Ser Pro Ile Ile Val Asp Ile Thr Lys Asp Thr Phe Tyr Lys
                        420 425 430
Gln Pro Met Phe Tyr His Leu Gly His Phe Ser Lys Phe Ile Pro Glu
                435 440 445
Gly Ser Gln Arg Val Gly Leu Val Ala Ser Gln Lys Asn Asp Leu Asp
        450 455 460
Ala Val Ala Leu Met His Pro Asp Gly Ser Ala Val Val Val Val Leu
465 470 475 480
Asn Arg Ser Ser Lys Asp Val Pro Leu Thr Ile Lys Asp Pro Ala Val
                                485 490 495
Gly Phe Leu Glu Thr Ile Ser Pro Gly Tyr Ser Ile His Thr Tyr Leu
                        500 505 510
Trp Arg¹ Arg Gln Arg_{(n′′′)}
                  515
wherein each _{n′′′} is independently 0 or 1, adn Arg¹ is either Arg
or His
```

1. A conjugate comprising a residue of a lysosomal enzyme moiety covalently attached, either directly or through a spacer moiety of one or more atoms, to a water-soluble, non-peptidic polymer.

2. The conjugate of claim 1, wherein the water-soluble, non-peptidic polymer is a linear water-soluble, non-peptidic polymer.

3. The conjugate of claim 1, wherein the water-soluble, non-peptidic polymer is a branched water-soluble, non-peptidic polymer.

4. The conjugate of claim 1, wherein the lysosomal enzyme moiety is recombinantly prepared.

5. The conjugate of claim 1, wherein the non-peptidic, water-soluble polymer is a poly(alkylene oxide).

6. The conjugate of claim 5, wherein the poly(alkylene oxide) is a poly(ethylene glycol).

7. The conjugate of claim 1, wherein the poly(ethylene glycol) is terminally capped with an end-capping moiety selected from the group consisting of alkoxy, substituted alkoxy, alkenoxy, substituted alkenoxy, alkynoxy, substituted alkynoxy, aryloxy and substituted aryloxy.

8. The conjugate of claim 1, wherein the non-peptidic, water-soluble polymer has a weight-average molecular weight in a range of from about 500 Daltons to about 100,000 Daltons.

9. The conjugate of claim 1, wherein the non-peptidic, water-soluble polymer has a weight-average molecular weight in a range of from about 2,000 Daltons to about 60,000 Daltons.

10. The conjugate of claim 1, wherein the conjugate has from one to four water-soluble polymers attached to the residue of the lysosomal enzyme moiety.

11. The conjugate of claim 10, wherein the conjugate has two water-soluble polymers attached to the residue of the lysosomal enzyme moiety.

12. The conjugate of claim 1, wherein the water-soluble, non-peptidic polymer is covalently attached at an amine terminus of the lysosomal enzyme moiety.

13. The conjugate of claim 1, wherein the water-soluble, non-peptidic polymer is covalently attached at an amine group of a lysine residue within the lysosomal enzyme moiety.

14. The conjugate of claim 1, wherein the water-soluble, non-peptidic polymer is covalently attached at a thiol group of a cysteine residue within the lysosomal enzyme moiety.

15. The conjugate of claim 1, wherein the lysosomal enzyme moiety is selected from the group consisting of glucocerebrosidase, laronidase, alpha-galactosidase-A, N-aceytlgalactosamine 4-sulfatase, alpha-glucosidase, and iduronate-2-sulfatase.

16. A pharmaceutical composition comprising a conjugate of claim 1 and a pharmaceutically acceptable excipient.

17. A method for making a conjugate comprising contacting, under conjugation conditions, a lysosomal enzyme moiety with a polymeric reagent.


SEQUENCE LISTING (6 sequences)

SEQ ID NO: 1 - protein, 499 aa, Homo sapiens
  MOD_RES (1)..(1): Methinone may be deleted
MARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQR

SEQ ID NO: 2 - protein, 498 aa, Homo sapiens
  MOD_RES (1)..(1): Methinone may be deleted
MARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 3 - protein, 499 aa, Homo sapiens
  MOD_RES (1)..(1): Methinone may be deleted
MARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQR

SEQ ID NO: 4 - protein, 498 aa, Homo sapiens
  MOD_RES (1)..(1): Methinone may be deleted
MARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 5 - protein, 537 aa, Homo sapiens
  MOD_RES (1)..(1): Methinone may be deleted
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQR

SEQ ID NO: 6 - protein, 517 aa, Homo sapiens
  MOD_RES (1)..(1): Methinone may be deleted
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQR